(19) United States
(12) Patent Application Publication
(10) Pub. No.: US 2013/0138159 A1
(43) Pub. Date: May 30, 2013

(54) Polyaxial bone anchor having an open retainer with conical, cylindrical or curvate capture

(71) Applicant: Roger P. Jackson, Prairie Village, KS (US)

(72) Inventor: Roger P. Jackson, Prairie Village, KS (US)

(21) Appl. No.: 13/694,956

(22) Filed: Jan. 22, 2013

Related U.S. Application Data
(62) Division of application No. 12/925,342, filed on Oct. 19, 2010.
Application No. 12/925,342 is a continuation-in-part of application No. 12/804,580, filed on Jul. 23, 2010, now Pat. No. 8,394,133.
Application No. 12/804,580 is a continuation of application No. 11/522,503, filed on Sep. 14, 2006, now Pat. No. 7,766,915.
Application No. 11/522,503 is a continuation-in-part of application No. 11/024,543, filed on Dec. 20, 2004, now Pat. No. 7,204,838.
Application No. 12/925,342 is a continuation-in-part of application No. 12/154,460, filed on May 23, 2008, now Pat. No. 8,257,396.
Application No. 12/154,460 is a continuation-in-part of application No. 11/140,343, filed on May 27, 2005, now Pat. No. 7,776,067.
Application No. 11/140,343 is a continuation-in-part of application No. 10/651,003, filed on Aug. 28, 2003, now Pat. No. 8,137,386.
Application No. 12/925,342 is a continuation-in-part of application No. 12/011,048, filed on Jan. 24, 2008, now Pat. No. 8,241,333.
Application No. 12/011,048 is a continuation of application No. 10/650,910, filed on Aug. 28, 2003, now Pat. No. 7,322,981.
(60) Provisional application No. 61/279,383, filed on Oct. 20, 2009.
(60) Provisional application No. 60/931,362, filed on May 23, 2007.

(51) Int. Cl. A61B 17/86

(52) U.S. Cl. CPC A61B 17/8605
USPC 606/305

(57) ABSTRACT

Polyaxial bone anchor assemblies include a shank having an upper portion and a retainer for holding the shank upper portion in a receiver. The shank upper portion retainer interface is one of conical, cylindrical or curvate and may further include a radial ridge or undercut. The assemblies may include compression inserts.

### CROSS-REFERENCE TO RELATED APPLICATIONS

This application is a Divisional of U.S. application Ser. No. 12/925,342, filed Oct. 17, 2010 that claimed the benefit of U.S. Provisional Pat. App. Ser. No. 61/279,383, filed Oct. 20, 2009, both of the disclosures of which are incorporated by reference herein. U.S. application Ser. No. 12/925,342 was a continuation-in-part of U.S. patent application Ser. No. 12/804,580 filed Jul. 23, 2010 that is a continuation of U.S. patent application Ser. No. 11/522,503 filed Sep. 14, 2006, now U.S. Pat. No. 7,766,915, that is a continuation-in-part of U.S. patent application Ser. No. 11/024,543 filed Dec. 20, 2004, now U.S. Pat. No. 7,204,838, all of the disclosures of which are incorporated by reference herein. U.S. application Ser. No. 12/925,342 was also a continuation-in-part of U.S. patent application Ser. No. 12/154,460 filed May 23, 2008 that claims the benefit of U.S. Prov. Pat. App. Ser. No. 60/931,362 filed May 23, 2007 and was a continuation-in-part of U.S. patent application Ser. No. 11/140,343 filed May 27, 2005 and a continuation-in-part of U.S. patent application Ser. No. 10/651,003 filed Aug. 28, 2003, all of the disclosures of which are incorporated by reference herein. U.S. application Ser. No. 12/925,342 was also a continuation-in-part of U.S. patent application Ser. No. 12/011,048 filed Jan. 24, 2008 that is a continuation of U.S. patent application Ser. No. 10/650,910 filed Aug. 28, 2003, now U.S. Pat. No. 7,322,981, all of the disclosures of which are incorporated by reference herein.

### BACKGROUND OF THE INVENTION

The present invention is directed to polyaxial bone screws for use in bone surgery, particularly spinal surgery and particularly to such screws with or without pressure inserts.

Bone screws are utilized in many types of spinal surgery in order to secure various implants to vertebrae along the spinal column for the purpose of stabilizing and/or adjusting spinal alignment. Although both closed-ended and open-ended bone screws are known, open-ended screws are particularly well suited for connections to rods and connector arms, because such rods or arms do not need to be passed through a closed bore, but rather can be laid or urged into an open channel within a receiver or head of such a screw.

Typical open-ended bone screws include a threaded shank with a pair of parallel projecting branches or arms which form a yoke with a U-shaped slot or channel to receive a rod. Hooks and other types of connectors, as are used in spinal fixation techniques, may also include open ends for receiving rods or portions of other structure.

A common mechanism for providing vertebral support is to implant bone screws into certain bones which then in turn support a longitudinal structure such as a rod, or are supported by such a rod. Bone screws of this type may have a fixed head or receiver relative to a shank thereof. In the fixed bone screws, the rod receiver head cannot be moved relative to the shank and the rod must be favorably positioned in order for it to be placed within the receiver head. This is sometimes very difficult or impossible to do. Therefore, polyaxial bone screws are commonly preferred.

Open-ended polyaxial bone screws allow rotation of the head or receiver about the shank until a desired rotational position of the head is achieved relative to the shank. Thereafter, a rod or other longitudinal connecting member can be inserted into the head or receiver and eventually the receiver is locked or fixed in a particular position relative to the shank. During the rod implantation process it is desirable to utilize bone screws or other bone anchors that have components that remain within the bone screw and further remain properly aligned during what is sometimes a very lengthy, difficult procedure.

### SUMMARY OF THE INVENTION

A polyaxial bone screw assembly according to the invention includes a shank having an upper portion and a body for fixation to a bone; a head or receiver defining an open channel; and, an open ring-like retainer for slidingly and pivotally holding the upper portion in the receiver. In some embodiments of the invention, the assembly further includes at least one compression insert spaced above and apart from the retainer structure. The shank upper portion is bottom or up-loadable into the receiver, cooperates with the retainer, and has a top end which extends above a top surface of the retainer, the retainer having one of a frusto-conical, cylindrical or curvate inner surface frictionally engageable with a respective frusto-conical, cylindrical or curvate surface of the shank upper portion, the retainer located between the shank upper portion and the receiver and spaced below the compression insert in those embodiments that include such an insert. In embodiments having a compression insert, such insert typically includes arms defining a U-shaped channel for receiving a longitudinal connecting member.

Therefore, it is an object of some embodiments of the present invention to provide apparatus and methods directed to an open retainer configured to fixedly engage a shank upper portion and slidably engage a receiver so as to polyaxially articulate with the receiver until the receiver is fixed relative to the shank, when a desired configuration is acquired, while therebetween holding the shank upper portion in spaced relation with respect to the receiver. Furthermore, it is an object of the invention to provide apparatus and methods that are easy to use and especially adapted for the intended use thereof and wherein the tools are comparatively inexpensive to produce.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a partial exploded side elevational view of a polyaxial bone screw assembly according to the present invention including a shank, a receiver, a retainer, and a closure top and shown with a longitudinal connecting member in the form of a rod.

FIG. 2 is an enlarged top plan view of the shank of FIG. 1.

FIG. 3 is an enlarged and partial cross-sectional view taken along the line 3-3 of FIG. 2.

FIG. 4 is an enlarged top plan view of the retainer of FIG. 1.

FIG. 5 is an enlarged perspective view of the retainer of FIG. 1.

FIG. 6 is an enlarged side elevational view of the retainer of FIG. 1.

FIG. 7 is an enlarged cross-sectional view taken along the line 7-7 of FIG. 6.

FIG. 8 is an enlarged and partial side elevational view of the shank, retainer and receiver of FIG. 1, with portions broken away to show the detail thereof, showing an early stage of assembly thereof.

FIG. 9 is an enlarged and partial side elevational view, similar to FIG. 8, with portions broken away to show the detail thereof and showing a later stage of assembly of the shank, retainer and receiver.

FIG. 10 is an enlarged and partial side elevational view, similar to FIG. 9, with portions broken away to show the detail thereof and showing a later stage of assembly of the shank, retainer and receiver.

FIG. 11 is an enlarged and partial side elevational view, similar to FIG. 10, with portions broken away to show the detail thereof and showing the shank, retainer and receiver in an assembled configuration.

FIG. 12 is an enlarged and partial side elevational view, similar to FIG. 11, with portions broken away to show the detail thereof, showing a degree of pivoting of the shank and attached retainer with respect to the receiver.

FIG. 13 is an enlarged and partial side elevational view, similar to FIG. 12, with portions broken away to show the detail thereof and further showing the rod of FIG. 1 inserted into the receiver and engaging the shank.

FIG. 14 is a reduced and partial side elevational view, similar to FIG. 13 and further showing the closure of FIG. 1 in a stage of assembly with the remainder of the assembly of FIG. 1.

FIG. 15 is a reduced and partial side elevational view, similar to FIG. 14, with portions broken away to show the detail thereof and showing the closure mated to the receiver and in fixed engagement with the rod.

FIG. 16 is an enlarged and partial front elevational view of the assembly of FIG. 15, with portions broken away to show the detail thereof.

FIG. 17 is an exploded perspective view of a second, alternative embodiment of a polyaxial bone screw assembly according to the present invention including a shank, a receiver, a retainer, a compression insert and a closure top, and further shown with a longitudinal connecting member in the form of a rod.

FIG. 18 is an enlarged top plan view of the shank of FIG. 17.

FIG. 19 is an enlarged and partial cross-sectional view taken along the line 19-19 of FIG. 18.

FIG. 20 is an enlarged and partial perspective view of the shank of FIG. 17.

FIG. 21 is an enlarged top plan view of the retainer of FIG. 17.

FIG. 22 is a perspective view of the retainer of FIG. 17.

FIG. 23 is a side elevational view of the retainer of FIG. 17.

FIG. 24 is a cross-sectional view taken along the line 24-24 of FIG. 23.

FIG. 25 is an enlarged and partial side elevational view of the shank, retainer and receiver of FIG. 17, with portions broken away to show the detail thereof, showing an early stage of assembly thereof.

FIG. 26 is an enlarged top plan view of the compression insert of FIG. 17.

FIG. 27 is an enlarged and partial side elevational view of the shank, retainer and receiver of FIG. 17, shown assembled with portions broken away to show the detail thereof and also showing an early stage of assembly with the compression insert of FIG. 17.

FIG. 28 is an enlarged and partial side elevational view of the shank, retainer, receiver and compression insert of FIG. 17, shown assembled with portions broken away to show the detail thereof.

FIG. 29 is a cross-sectional view taken along the line 29-29 of FIG. 28.

FIG. 30 is an enlarged and partial side elevational view of the assembly of FIG. 17 shown fully assembled and locked in position.

FIG. 31 is a partial cross-sectional view taken along the line 31-31 of FIG. 30.

FIG. 32 is a reduced and partial perspective view of the assembly of FIG. 30.

FIG. 33 is an exploded perspective view of a third, alternative embodiment of a polyaxial bone screw assembly according to the present invention including a shank, a receiver, a retainer, a compression insert and a closure top.

FIG. 34 is an enlarged top plan view of the shank of FIG. 33.

FIG. 35 is a reduced and partial cross-sectional view taken along the line 35-35 of FIG. 34.

FIG. 36 is an enlarged top plan view of the retainer of FIG. 33.

FIG. 37 is an enlarged and partial perspective view of the retainer of FIG. 33 with portions broken away to show the detail thereof.

FIG. 38 is an enlarged cross-sectional view taken along the line 38-38 of FIG. 36.

FIG. 39 is a partial side elevational view of the shank, retainer and receiver of FIG. 33, with portions broken away to show the detail thereof, showing an early stage of assembly thereof.

FIG. 40 is a partial side elevational view, similar to FIG. 39, with portions broken away to show the detail thereof and showing a later stage of assembly of the shank, retainer and receiver.

FIG. 41 is an enlarged and partial side elevational view with portions broken away, similar to FIG. 40, showing only the shank and retainer.

FIG. 42 is an enlarged and partial side elevational view of the shank, retainer, receiver and closure top of FIG. 33, further shown with a longitudinal connecting member in the form of a rod, with portions broken away to show the detail thereof.

FIG. 43 is a partial cross-sectional view taken along the line 43-43 of FIG. 42.

FIG. 44 is a partial perspective view of the assembly of FIG. 33, shown with a longitudinal connecting member in the form of a rod and with the shank disposed at an obtuse angle with respect to the receiver.

FIG. 45 is an enlarged and partial side elevational view of the assembly as shown in FIG. 44, with portions broken away to show the detail thereof.

FIG. 46 is an exploded perspective view of a fourth, alternative embodiment of a polyaxial bone screw assembly according to the present invention including a shank, a receiver, a retainer, a compression insert and a closure top.

FIG. 47 is an enlarged and fragmentary elevational view of the shank of FIG. 46 showing a capture portion at an upper end thereof.

FIG. 48 is a top plan view of the shank of FIG. 47.

FIG. 49 is a fragmentary cross-sectional view of the shank taken along the line 49-49 of FIG. 48.

FIG. 50 is an enlarged, perspective view of the retainer of FIG. 46.

FIG. 51 is a top plan view of the retainer of FIG. 50.

FIG. 52 is a cross-sectional view taken along the line 52-52 of FIG. 51.

FIG. 53 is an enlarged and fragmentary side elevational view of the shank, retainer and receiver of FIG. 46, with portions broken away to show the detail thereof, showing the retainer positioned on the shank prior to securement of the shank and retainer within the receiver.

FIG. 54 is an enlarged and fragmentary side elevational view of the shank, retainer, receiver and compression insert of FIG. 46, shown assembled with portions broken away to show the detail thereof.

FIG. 55 is a fragmentary elevational view of an alternative shank with a modified capture structure formed at an upper end thereof.

FIG. 56 is a cross-sectional, fragmentary view of the shank as shown in FIG. 55.

FIG. 57 is an enlarged, perspective view of a retainer for use in association with the shank of FIG. 55.

FIG. 58 is a top plan view of the retainer of FIG. 57.

FIG. 59 is a cross-sectional view taken along the line 59-59 of FIG. 58.

FIG. 60 is an enlarged and fragmentary side elevational view of the shank of FIG. 55 and retainer of FIG. 57 positioned thereon prior to securement of the shank and retainer within the receiver of FIG. 46.

FIG. 61 is an enlarged and fragmentary side elevational view of the shank, retainer and receiver of FIG. 61 and a compression insert of FIG. 46, shown assembled with portions broken away to show the detail thereof.

FIG. 62 is a fragmentary and enlarged elevational view of an alternative shank with a modified capture structure formed at an upper end thereof, with portions broken away to show the detail thereof.

FIG. 63. a fragmentary and enlarged elevational view of an alternative shank with a modified capture structure formed at an upper end thereof.

FIG. 64 is a perspective view of a retainer for securement on the shank of FIG. 62.

FIG. 65 is a front elevational view of the retainer of FIG. 64 with portions broken away to show the detail thereof.

FIG. 66. a fragmentary and enlarged elevational view of an alternative shank with a modified capture structure formed at an upper end thereof.

FIG. 67 is a front elevational view, with portions broken away, of a retainer for securement on the shank of FIG. 66.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the bone attachment structures in actual use.

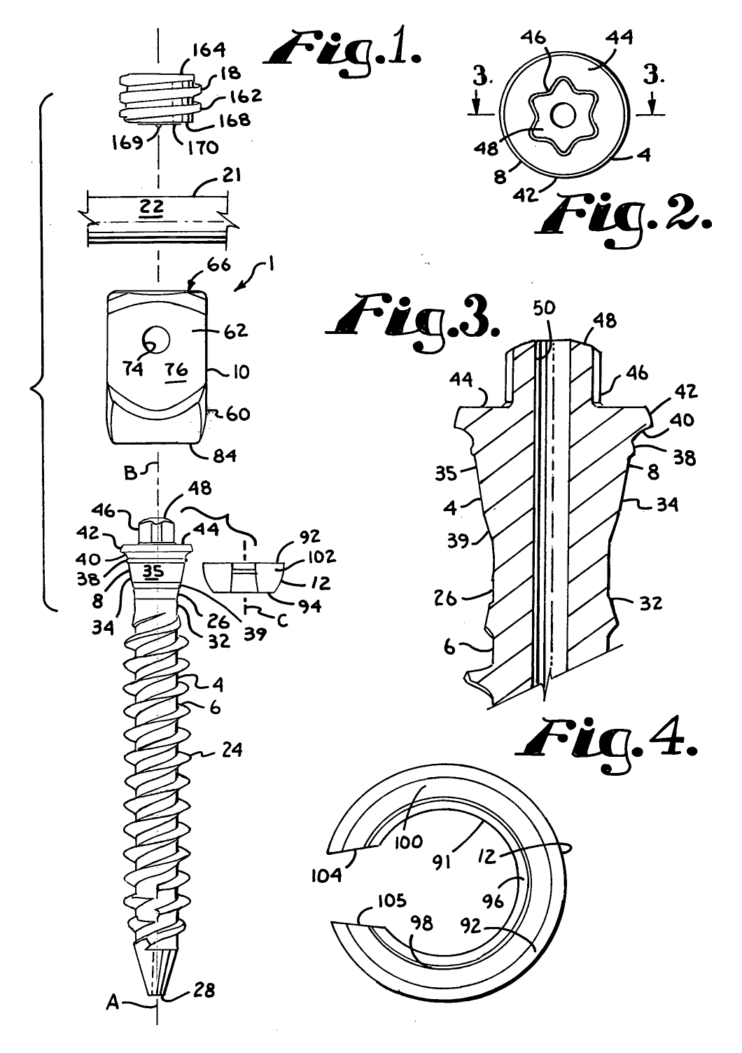

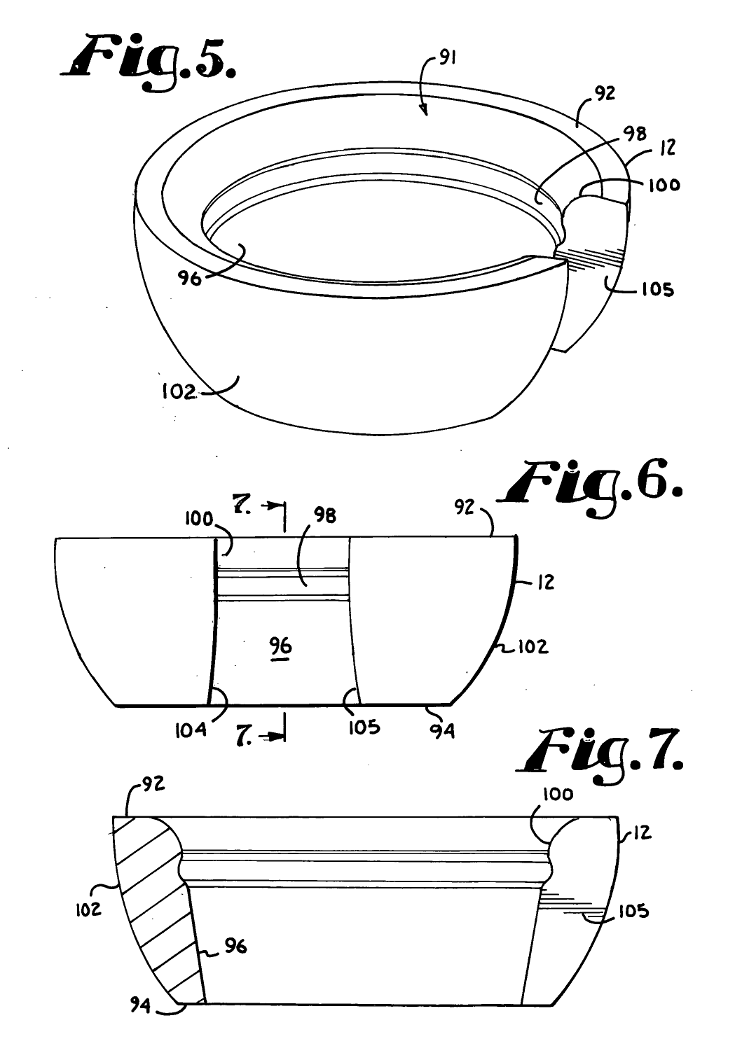

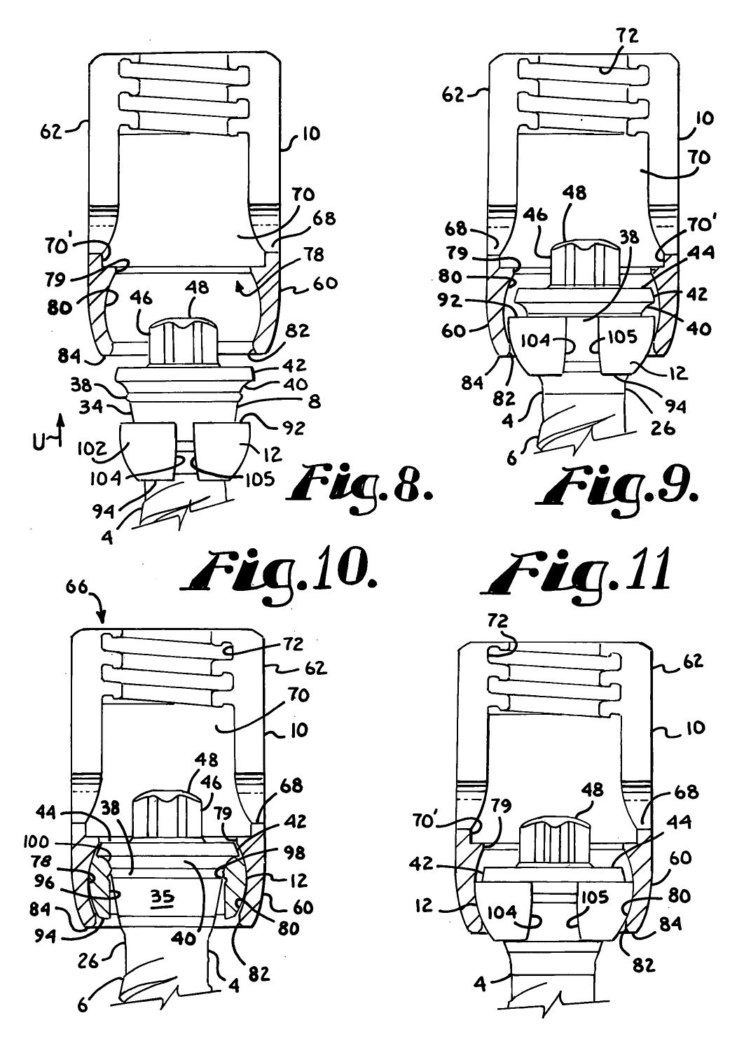

With reference to FIGS. 1-16 the reference number 1 generally represents a polyaxial bone screw apparatus or assembly according to the present invention. The assembly 1 includes a shank 4, that further includes a body 6 integral with an upwardly extending upper portion or capture structure 8; a receiver 10; and a retainer structure 12. The shank 4, receiver 10 and retainer structure 12 preferably are assembled prior to implantation of the shank body 6 into a vertebra 13. FIG. 1 further shows a closure structure 18 of the invention for capturing a longitudinal member, for example, such as the illustrated rod 21 which in turn engages an upper curved area of the shank upper portion 8 and biases the retainer structure 12 into fixed frictional contact with the receiver 10, so as to capture, and in some embodiments, fix the longitudinal connecting member 21 within the receiver 10 and thus fix the member 21 relative to the vertebra 13. The illustrated rod 21 is hard, stiff, non-elastic and cylindrical, having an outer cylindrical surface 22. In other embodiments, the rod 21 may be elastic, deformable and/or of a different cross-sectional geometry, as will be described in greater detail below. The upper curved area of the shank upper portion 8 is spaced above the retainer 12 and the retainer 12 is disposed between the shank upper portion 8 and the receiver 10. The receiver 10 and the shank 4 cooperate in such a manner that the receiver 10 and the shank 4 can be secured at any of a plurality of angles, articulations or rotational alignments relative to one another and within a selected range of angles both from side to side and from front to rear, to enable flexible or articulated engagement of the receiver 10 with the shank 4 until both are locked or fixed relative to each other near the end of an implantation procedure.

The shank 4, best illustrated in FIGS. 1-3, is elongate, with the shank body 6 having a helically wound bone implantable thread 24 (single or dual lead thread form) extending from near a neck 26 located adjacent to the upper portion or capture structure 8, to a tip 28 of the body 6 and extending radially outwardly therefrom. During use, the body 6 utilizing the thread 24 for gripping and advancement is implanted into a vertebra 13 leading with the tip 28 and driven down into the vertebra with an installation or driving tool (not shown), so as to be implanted in the vertebra to near the neck 26, as more fully described in the paragraphs below. The shank 4 has an elongate axis of rotation generally identified by the reference letter A.

The neck 26 extends axially upward from the shank body 6. The neck 26 may be of the same or slightly reduced radius as compared to an adjacent upper end or top 32 of the body 6 where the thread 24 terminates. Further extending axially and outwardly from the neck 26 is the shank upper portion 8 that provides a connective or capture apparatus disposed at a distance from the upper end 32 and thus at a distance from a vertebra 13 when the body 6 is implanted in such vertebra.

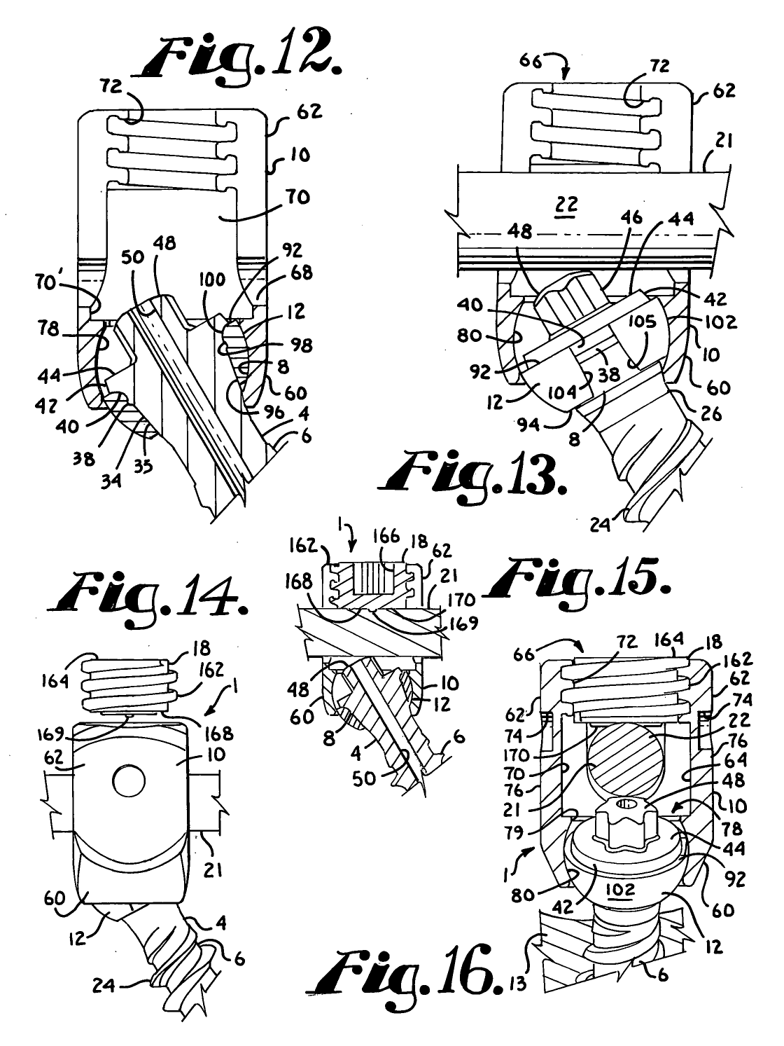

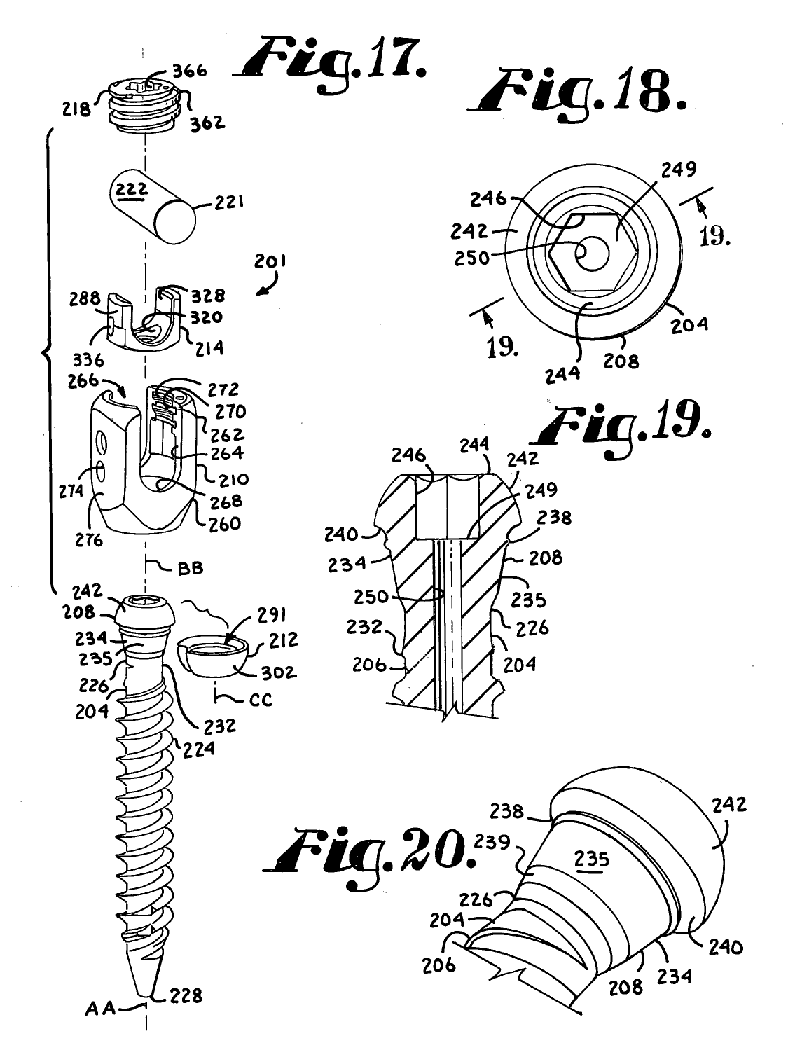

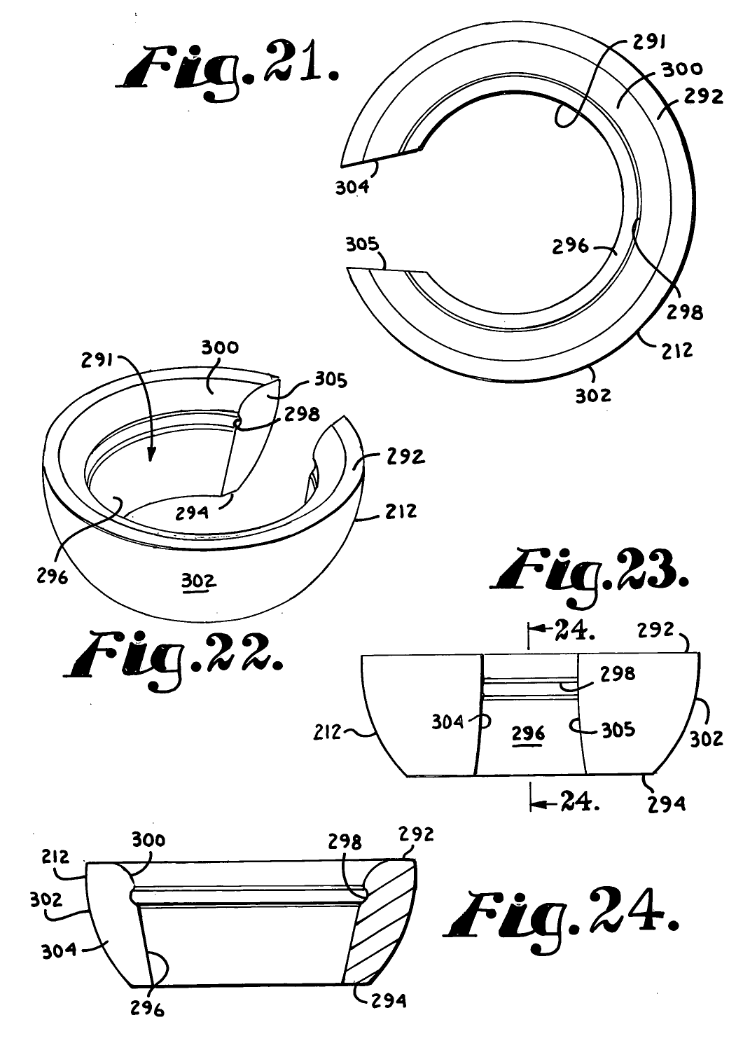

The shank upper portion 8 is configured for a fixed connection between the shank 4 and the retainer structure 12 and a pivotable connection between the shank 4/retainer structure 12 combination and the receiver 10 prior to fixing of the shank in a desired position with respect to the receiver 10. The upper portion 8 generally includes a substantially frusto-conical lower body 34 having a frusto-conical surface 35. The body 34 may include more than one frusto-conical surfaces graduating from the neck 26 to a convex, radially extending ring-like rib or ridge 38. The illustrated body 34 includes a lower frusto-conical surface 39 located near the neck 26 that is adjacent to the frusto-conical surface 35. The ridge 38 is sized and shaped to be received in a closely mating groove of the retainer 12 as will be described in greater detail below, the rib and groove combination providing for secure engagement of the retainer 12 against the shank upper portion 8 at a desired location and orientation, prohibiting upward and downward movement of the retainer 12 along the shank axis A. A curved, concave radially extending collar or flange 40 is located adjacent the rib 38 and extends outwardly from the axis A to an outer surface 42 that is illustrated as semi-spherical in form, curving inwardly toward the axis A in an upward direction toward a substantially planar upper surface 44, the surface 44 being annular and disposed substantially perpendicular to the axis A. In some embodiments, the surface 42 may be frusto-conical or cylindrical in form. The curved collar or flange 40 is sized and shaped to readily assemble with and closely receive a curved upper portion of the retainer 12 as will be described in greater detail below. An external tool engagement drive feature or structure 46 extends upwardly along the axis A away from the upper surface 44 and is illustrated as a multi-faceted star-shape structure sized and shaped to mate with a socket driving tool (not shown) having an internal drive configured to fit about the tool engagement structure 46 for both driving and rotating the shank body 6 into the vertebra. Although a star-shaped drive 46 is illustrated, the drive 46 may have other shapes, including, but not limited to, a hex-shaped form; or an internal drive may be utilized. A top surface 48 of the drive structure 46 is preferably curved, radiused or dome shaped as shown in the drawings, for contact and positive mating engagement with the surface 22 of the rod 21 when the bone screw assembly 1 is fully assembled, as shown, for example, in FIGS. 15 and 16 and in any pivotal alignment of the shank 4 relative to the receiver 10. In the illustrated embodiment, the surface 48 is smooth. While not required in accordance with the practice of the invention, the surface 48 may be scored or knurled to further increase frictional positive mating engagement between the surface 48 and the rod 21. The shank 4 shown in the drawings is cannulated, having a small central bore 50 extending an entire length of the shank 4 along the axis A. The bore 50 is defined by an inner cylindrical wall of the shank 4 and has a circular opening at the shank tip 28 and an upper opening communicating with the external drive top surface 48. The bore 50 is coaxial with the threaded body 6 and the upper portion 8. The bore 50 provides a passage through the shank 4 interior for a length of wire (not shown) inserted into the vertebra 13 prior to the insertion of the shank body 6, the wire providing a guide for insertion of the shank body 6 into the vertebra.

To provide a biologically active interface with the bone, the threaded shank body 6 may be coated, perforated, made porous or otherwise treated. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄)₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

With particular reference to FIGS. 1, 8 and 16, the receiver 10 has a generally U-shaped appearance with a discontinuous partially cylindrical and partially spherical inner profile and a partially curved and partially faceted outer profile. The receiver 10 has an axis of rotation B that is shown in FIG. 1 as being aligned with and the same as the axis of rotation A of the shank 4, such orientation being desirable during assembly of the receiver 10 with the shank 4 and the retainer 12. After the receiver 10 is pivotally attached to the shank 4, and the assembly 1 is implanted in a vertebra 13, the axis B is typically disposed at an angle with respect to the axis A, as shown, for example in FIGS. 12-16.

The receiver 10 includes a base 60 integral with a pair of opposed upstanding arms 62 forming a cradle and defining a channel 64 between the arms 62 with an upper opening, generally 66, and a lower seat 68, the channel 64 having a width for operably snugly receiving the rod 21 between the arms 62. Each of the arms 62 has an interior surface 70 that defines the inner cylindrical profile and includes a partial helically wound guide and advancement structure 72. In the illustrated embodiment, the guide and advancement structure 72 is a partial helically wound interlocking flangeform configured to mate under rotation with a similar structure on the closure structure 18, as described more fully below. However, it is foreseen that the guide and advancement structure 72 could alternatively be a square-shaped thread, a buttress thread, a reverse angle thread or other thread-like or non-thread-like helically wound discontinuous advancement structure for operably guiding under rotation and advancing the closure structure 18 downward between the arms 62, as well as eventual torquing when the closure structure 18 abuts against the rod 21 in some embodiments or abuts against a compression insert in other embodiments.

An opposed pair of tool receiving and engaging apertures 74 are formed on outer surfaces 76 of the arms 62. The apertures 74 may be used for holding the receiver 10 during assembly with the shank 4 and the retainer structure 12, during the implantation of the shank body 6 into a vertebra (not shown) and assembly with the rod 21 and the closure structure 18. It is foreseen that tool receiving grooves or apertures may be configured in a variety of shapes and sizes and be disposed at other locations on the receiver arms 62.

Communicating with and located beneath the channel 64 of the receiver 10 at the base portion 60 thereof is a chamber or cavity, generally 78, defined in part by an inner substantially cylindrical surface 79 and a substantially spherical seating surface portion 80. The cavity 78 is also defined in part by a cylindrical inner wall 70′ located above and adjacent to the cylindrical surface 79, the wall 70′ being formed by the joining of the inner cylindrical walls 70 of each of the arms 62, the wall 70′ providing a support for the channel seat 68. The cylindrical surface 79 is adjacent to and disposed between the cylindrical wall 70′ and the substantially spherical seating surface 80. The surface 80 is sized and shaped for slidably mating with the retainer structure 12 and ultimately frictionally mating therewith as will be described in greater detail below. The spherical surface portion 80 communicates with a lower opening neck 82 that communicates with both the cavity 78 and a receiver lower exterior or bottom 84 of the base 60. The neck 82 is substantially coaxially aligned with respect to the rotational axis B of the receiver 10. The lower neck 82 is also sized and shaped to be smaller than an outer radial dimension of the retainer structure 12 when the retainer 12 is fixed to the shank upper portion 8, so as to form a restriction to prevent the structure 12 and attached shank portion 8 from passing through the cavity 78 and out the lower exterior 84 of the receiver 10 during operation thereof.

The retainer structure or retainer 12 is used to capture the shank upper portion 8 and retain the upper portion 8 within the receiver 10 as well as swivel or articulate with respect to the receiver 10. The retainer 12, best illustrated in FIGS. 1 and 4-7 has an operational central axis that is the same as the rotational axis A associated with the shank 4, but when the retainer 12 is separated from the shank 4, the axis of rotation is identified as axis C, as shown in FIG. 1. The retainer 12 is open, having a through slit and a central bore 91 that passes entirely through the retainer 12 from a top surface 92 to a bottom surface 94 thereof. Both the top surface 92 and the bottom surface 94 are substantially planar and disposed perpendicular to the axis C. A first inner frusto-conical surface 96 defines a substantial portion of the bore 91, the surface 96 being adjacent to the bottom surface 94. The surface 96 is sized and shaped to be closely received about the shank surface 35 when the retainer 12 and the shank upper portion 8 are frictionally engaged within the receiver 10. A groove 98 extends radially outwardly from the axis C and into the surface 96, the groove 98 being sized and shaped to closely receive the rib or ridge 38 of the shank upper portion 8. A convex radiused surface portion 100 extends between the groove 98 and the top surface 92. The outwardly curved surface portion 100 is sized and shaped to be closely received by and mate with the concave flanged collar 40 of the shank upper portion 8 during installation of the retainer 12 on the shank upper portion 8 within the receiver 10 as will be described in greater detail below.

The retainer 12 also has a radially outer partially spherically shaped surface 102 running between the top surface 92 and the bottom surface 94, the surface 102 being sized and shaped to mate with the partially spherical shaped seating surface 80 of the receiver 10. The surface 102 includes an outer radius that is larger than a radius of the neck lower opening 82 of the receiver 10 when the retainer 12 is in a neutral, non-compressed state, thereby prohibiting the retainer 12 and the shank upper portion 8 from passing through the neck 82 once the retainer 12 is fixed to the shank upper portion 8 within the receiver cavity 78. Although not required, it is foreseen that the outer partially spherically shaped surface 102 may be a high friction surface such as a knurled surface or the like.

As previously noted, the retainer 12 is ring-like and also open, having a slit or gap formed by spaced end surfaces 104 and 105. In the illustrated embodiments, the surfaces 104 and 105 substantially face one another and are oriented at a slight angle with respect to one another, the surfaces 104 and 105 being slightly closer together at the outer surface 102 than at the inner frusto-conical surface 96. In other embodiments of the invention, the surfaces 104 and 105 may be parallel to one another. The illustrated surfaces 104 and 105 each run substantially parallel to the axis C. In other embodiments, one or both surfaces may be at an obtuse angle with respect to the axis C. The surfaces 104 and 105 are sized and shaped for allowing adequate clearance between the surfaces 104 and 105 when the retainer 12 is squeezed about the shank neck 26 and loaded with the shank upper portion 8 into the receiver 10 as shown in FIGS. 8-10 and described in greater detail below. Once installed and locked into position, the retainer 12 closely grips the shank at the frusto-conical surface 35 and the rib 38, the surfaces 104 and 105 being in a substantially neutral, spaced position, with the inner frusto-conical surface 96 providing a substantially even and uniform gripping surface between the shank 4 and the receiver 10 at the spherical seating surface 80 when force is directed onto the shank domed surface 48 by the closure structure 18 pressing on the rod 21. The frictionally mating radial rib 38 and groove 98 combination ensure a desired position and orientation of the retainer 12 with respect to the shank upper portion 8 regardless of other forces placed upon the retainer 12 within the receiver 10.

The longitudinal connecting member 21 that is utilized with the assembly 1 can be any of a variety of implants utilized in reconstructive spinal surgery, and is illustrated as a cylindrical elongate structure or rod having the cylindrical surface 22 of uniform diameter and having a generally smooth surface. The longitudinal connecting member 21 may be made from metal, metal alloys or other suitable materials, including plastic polymers such as polyetheretherketone (PEEK), ultra-high-molecular weight-polyethylene (UHMWP), polyurethanes and composites. The illustrated longitudinal connecting member 21 is preferably sized and shaped to snugly seat near the bottom of the channel 64 of the receiver 10 and, during normal operation, is positioned slightly above the bottom of the channel 64. In particular, the longitudinal connecting member 21 normally directly or abuttingly engages the domed shank top surface 48 and is biased against the surface 48, consequently biasing the shank 4 downwardly in a direction toward the base 60 of the receiver 10 when the assembly 1 is fully assembled. For this to occur, the shank top surface 48 must extend at least slightly into the space of the channel 64 when the retainer structure 12 is snugly seated against the receiver seating surface 80. The shank 4 and the retainer 12 are locked or held in position relative to the receiver 10 by the longitudinal connecting member 21 firmly pushing downward on the shank top surface 48 as illustrated, for example, in FIGS. 15 and 16.

Longitudinal connecting members for use with the bone screws of the invention may take a variety of shapes, including but not limited to rods or bars of oval, rectangular or other curved or polygonal cross-section. Furthermore, the connector 21 may be a component of a longer overall dynamic stabilization connecting member, with cylindrical or bar-shaped portions sized and shaped for being received by the receiver 10 that may have a U-, rectangular or other shaped channel for closely receiving the longitudinal connecting member. The connector 21 may be integral or otherwise fixed to bendable or damping components that are sized and shaped to be located between adjacent pairs of bone screw assemblies 1, for example. Such a rod or bar component may be made from a variety of materials including metal, metal alloys or other suitable materials, including, but not limited to plastic polymers such as polyetheretherketone (PEEK), ultra-high-molecular weight-polyethylene (UHMWP), polyurethanes and composites, including composites containing carbon fiber, as well as resorbable materials, such as polylactic acids.

With reference to FIGS. 1 and 14-16, the closure structure or closure top 18 shown with the assembly 1 is rotatably received between the spaced arms 62. It is noted that the closure 18 can be any of a variety of different types of closure structures for use in conjunction with the present invention with suitable mating structure on the upstanding arms 62. It is also foreseen that the closure top could be a twist-in or slide-in closure structure. The illustrated closure structure 18 is substantially cylindrical and includes an outer helically wound guide and advancement structure 162 in the form of a flange form that operably joins with the guide and advancement structure 72 disposed on the arms 62 of the receiver 10. The flange form utilized in accordance with the present invention may take a variety of forms, including those described in Applicant's U.S. Pat. No. 6,726,689, which is incorporated herein by reference. It is also foreseen that according to the invention the closure structure guide and advancement structure could alternatively be a buttress thread, a square thread, a reverse angle thread or other thread like or non-thread like helically wound advancement structure for operably guiding under rotation and advancing the closure structure 18 downward between the arms 62 and having such a nature as to resist splaying of the arms 62 when the closure structure 18 is advanced into the receiver channel 64. The illustrated closure structure 18 also includes a top surface 164 with an internal drive 166 in the form of an aperture that is illustrated as a star-shaped internal drive such as that sold under the trademark TORX, or may be, for example, a hex drive, or other internal drives such as slotted, tri-wing, spanner, two or more apertures of various shapes, and the like. A driving tool (not shown) sized and shaped for engagement with the internal drive 166 is used for both rotatable engagement and, if needed, disengagement of the closure 18 from the receiver arms 62. It is also foreseen that the closure structure 18 may alternatively include a break-off head designed to allow such a head to break from a base of the closure at a preselected torque, for example, 70 to 140 inch pounds. Such a closure structure would also include a base having an internal drive to be used for closure removal. A base or bottom surface 168 of the closure is illustrated as planar, and further includes an optional point 169 and rim 170 for engagement with the surface 22 of the rod 21 in certain embodiments of the invention. The closure top 18 may further include a cannulation through bore (not shown) extending along a central axis thereof and through the top and bottom surfaces thereof. Such a through bore provides a passage through the closure 18 interior for a length of wire (not shown) inserted therein to provide a guide for insertion of the closure top into the receiver arms 62.

With particular reference to FIGS. 15 and 16, when used with the hard, stiff rod 21, the closure top 18 engages and locks the rod 21 with the point 169 and the rim 170 penetrating into the rod surface 22. In other embodiments of the invention, the planar bottom surface 168 may engage a pressure or compression insert to press such insert down into locking engagement with the shank 4 with or without locking engagement with the rod 21. Thus, in some embodiments of the invention, (as will be described in greater detail with respect to the assembly 201), the bone screw assembly cooperates with a rod, cord, cable or other longitudinal connecting member to capture such connecting member within the receiver, but to allow the connector some freedom of movement within the receiver 10. In such applications, elastic spacers can be positioned around the connecting member and between the receivers. A closure top/insert combination may also be desirable when the connecting member is made from a deformable plastic. In such embodiments, the closure bottom surface may engage and frictionally hold the connecting member in place, but the polyaxial mechanism may be firmly locked in place by the closure directly engaging and pressing upon the compression insert that in turn presses on the shank upper portion, desirably holding, but not over-stressing the longitudinal connecting member at the cite of engagement with the bone screw. In the illustrated assembly 1, the hard, inelastic rod 21 is cradled by the receiver 10 and directly engages the shank upper surface 48 and pushes downwardly on the shank upper portion 8 by pressure from the closure structure 18, consequently pressing the shank 4 downwardly in a direction toward the base 60 of the receiver 10 when the assembly 1 is fully assembled, ultimately pressing the retainer 12 into frictional engagement with the receiver seating surface 80, thereby locking the polyaxial mechanism of the bone screw assembly 1.

With particular reference to FIGS. 1 and 8-10, prior to the polyaxial bone screw assembly 1 being placed in use according to the invention, the surfaces 104 and 105 of the retainer 12 are moved or pulled away from one another, widening the space or gap therebetween and allowing the retainer 12 to be slipped over and around the shank 4 at or near the neck 26. With reference to FIG. 8, the retainer structure 12 is then squeezed with the surfaces 104 and 105 being moved close together and a width and outer circumference of the retainer 12 being compressed or minimized to allow for bottom loading of both the compressed retainer 12 and the shank upper portion 8 into the receiver 10 in a direction indicated by an arrow U, uploading the retainer 12 and shank upper portion 8 through the lower opening defined by the neck 82, as shown in FIG. 9. Alternatively, in some embodiments, the tip 28 of the shank 6 is inserted into the through bore 91 of the retainer structure 12 and the structure 12 is moved or threaded up the shaft 6 of the shank 4 to a position about or near the neck 26 and the shank upper portion 8, such gap between the surfaces 104 and 105 allowing for such movement with the surfaces 104 and 105 being movable away from one another to provide clearance about the shank thread 24, if necessary. Thereafter, the retainer 12 is squeezed about the shank 4 and uploaded into the receiver 10 as previously described herein.

With reference to FIG. 8 and particularly to FIG. 9, the retainer structure 12, now substantially disposed in the receiver 10 is released from compression, allowing the return of the original or neutral spaced relation between the surfaces 104 and 105 as shown in FIG. 6. The retainer structure 12 is now captured within the receiver 10 with the outer spherical surface 102 in sliding engagement with the receiver inner spherical seating surface 80. The shank upper portion surface 42 is desirably configured such that a majority of the shank upper portion 8 is captured by the cylindrical surface 79 and prohibited from traveling upwardly into the channel 64 during assembly. The shank upper portion 8 is then pulled downwardly toward the receiver base neck 82, with the resilient retainer 12 sliding upwardly along the shank surface 35. As the shank upper portion 8 moves downwardly, the curved retainer surface 100 contacts the rib 38 and is pushed radially outwardly and then upper portions of the surface 100 slide along the surface 40, until the rib 38 is received into the groove 98, at which time the retainer 12 resiliently moves into position about the rib 38 with the surface 40 fully engaging the surface 100 and the surface 96 also frictionally engaging the shank frusto-conical surface 35.

Preferably, the shank 4, retainer 12 and receiver 10 are assembled at a factory setting that includes tooling for holding and alignment until the rib 38 is received in the groove 98. Permanent, rigid engagement of the shank upper portion 8 to the retainer structure 12 may be further supported by the use of adhesive, a spot weld, a deformation, or the like. At this time the shank 4 and the attached retainer 12 are fixed or coupled to one another and both are in pivotal, swivelable engagement with respect to the receiver 10. The retainer 12 is in slidable engagement with the receiver curvate seating surface 80. The shank body 6 can be rotated through a substantial angular rotation relative to the receiver 10, both from side to side and from front to rear so as to substantially provide a universal or ball joint.

The bone screw assembly made up of the assembled shank 4, receiver 10 and retainer 12 is then normally screwed into a bone, such as the vertebra 13, by rotation of the shank 4 using a suitable driving tool (not shown) that operably drives and rotates the shank body 6 by engagement thereof at the external drive 46. Specifically, the vertebra 13 may be pre-drilled to minimize stressing the bone and have a guide wire (not shown) inserted therein to provide a guide for the placement and angle of the shank 4 with respect to the vertebra. A further tap hole may be made using a tap with the guide wire as a guide. Then, the bone screw assembly is threaded onto the guide wire utilizing the cannulation bore 50 by first threading the wire into the opening at the bottom 28 and then out of the top opening at the drive feature 46. The shank 4 is then driven into the vertebra using the wire as a placement guide. It is foreseen that the bone screw assembly 1, the rod 21 (also having a central lumen in some embodiments) and the closure top 18 (also with a central bore) can be inserted in a percutaneous or minimally invasive surgical manner, utilizing guide wires.

With reference to FIGS. 13-16, the rod 21 is eventually positioned in an open or percutaneous manner in cooperation with the at least two bone screw assemblies 1. The closure structure 18 is then inserted into and advanced between the arms 62 of each of the receivers 10. The closure structure 18 is rotated, using a tool engaged with the inner drive 166 until a selected pressure is reached at which point the rod 21 engages the domed surface 48 of the shank 4 and the rod is urged toward, but not in contact with the lower seat 68 of the receiver 10 that defines the channel 64. For example, about 80 to about 120 inch pounds pressure may be required for fixing the bone screw shank 6 with respect to the receiver 10.

As the closure structure 18 rotates and moves downwardly into the respective receiver 10, the point 169 and rim 170 engage and penetrate the rod surface 22, the closure structure 18 pressing against and biasing the rod 21 into engagement with the shank surface 48 that urges the shank upper portion 8 toward the retainer 12 and, in turn, the structure 12 in a direction toward the base 60 of the receiver 10, so as to frictionally seat the spherical surface 102 against the internal spherical seating surface 80 of the receiver 10, also fixing the shank 4 and the retainer 12 in a selected, rigid position relative to the receiver 10. At this time it is also possible for the retainer 12 to expand somewhat for an even tighter fit in the receiver cavity lower seat 80.

If removal of the rod 21 from any of the bone screw assemblies 1 is necessary, or if it is desired to release the rod 21 at a particular location, disassembly is accomplished by using the driving tool (not shown) that mates with the internal drive 166 on the closure structure 18 to rotate and remove such closure structure from the cooperating receiver 10. Disassembly is then accomplished in reverse order to the procedure described previously herein for assembly.

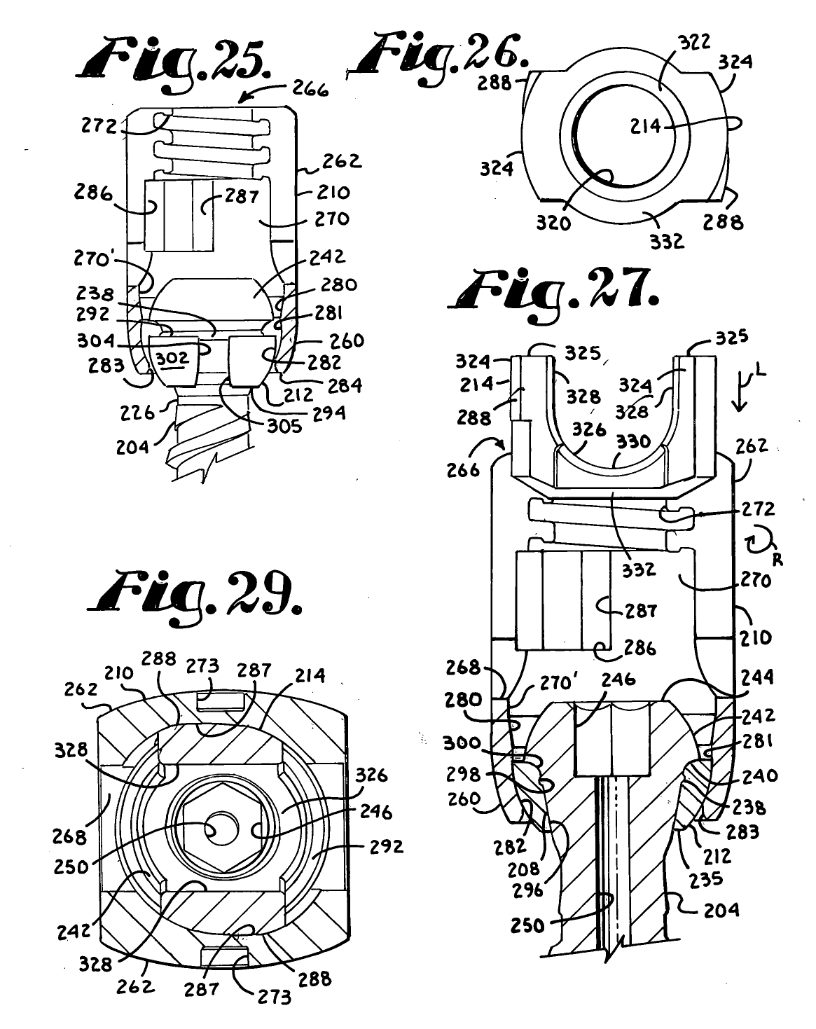

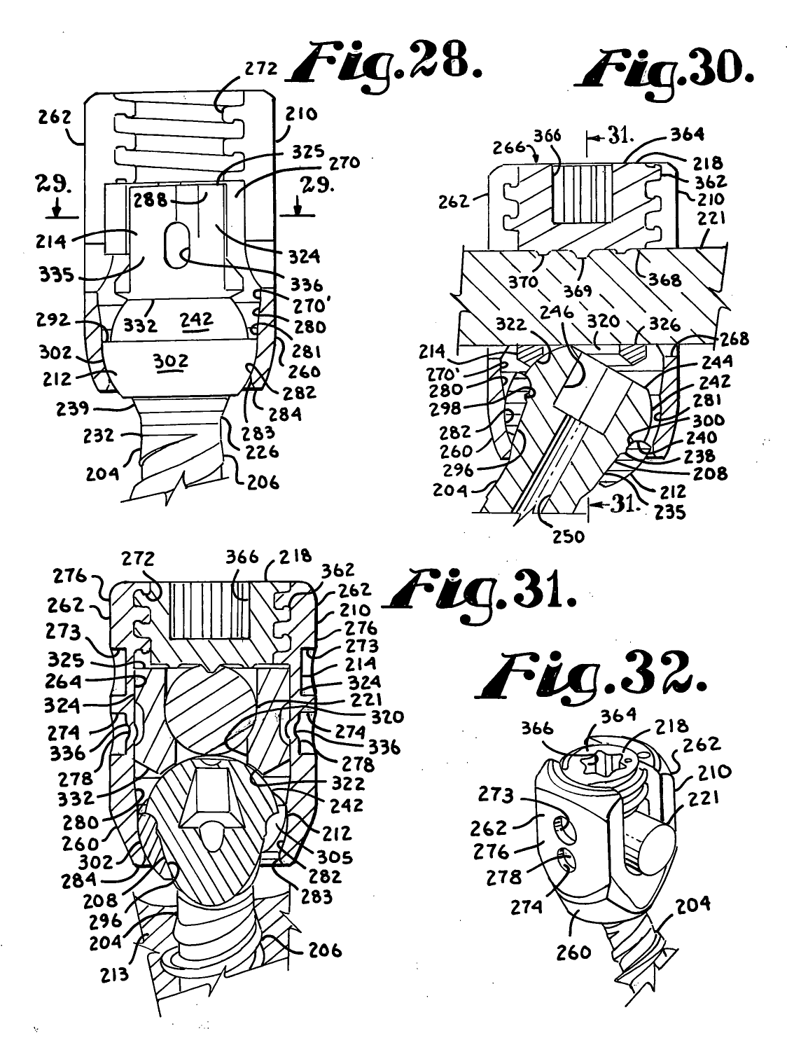

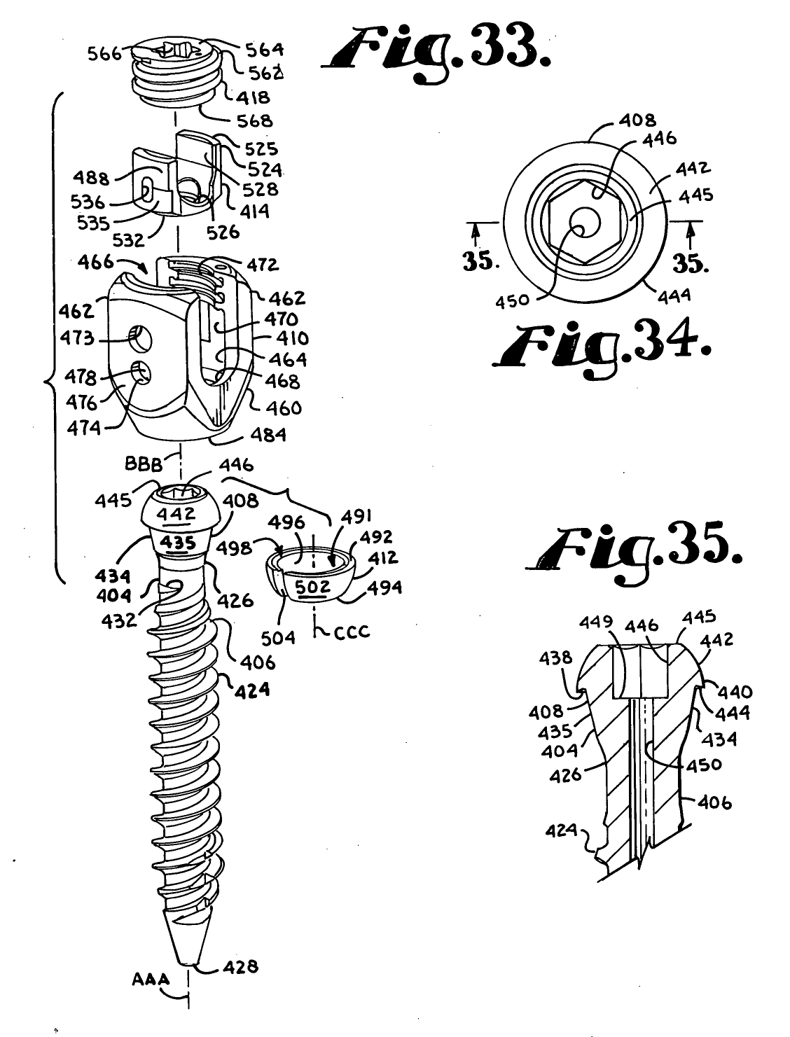

With reference to FIGS. 17-32, a second embodiment of a polyaxial bone screw assembly according to the invention, generally 201, includes a shank 204 having a body 206 and an upper portion 208, a receiver 210, a retainer 212, a compression insert 214 and a closure structure 218 and is shown with a longitudinal connecting member in the form of a hard, inelastic, substantially non-deformable rod 221 having a substantially cylindrical outer surface 222.

The shank 204, best illustrated in FIGS. 17-20, is elongate, with the shank body 206 having a helically wound bone implantable thread 224 (single or dual lead thread form) extending from near a neck 226 located adjacent to the upper portion or capture structure 208, to a tip 228 of the body 206 and extending radially outwardly therefrom. During use, the body 206 utilizing the thread 224 for gripping and advancement is implanted into a vertebra 213 leading with the tip 228 and driven down into the vertebra with an installation or driving tool (not shown), so as to be implanted in the vertebra to near the neck 226. The shank 204 has an elongate axis of rotation generally identified by the reference letter AA.

The neck 206 extends axially upward from the shank body 206. The neck 226 may be of the same or slightly reduced radius as compared to an adjacent upper end or top 232 of the body 206 where the thread 224 terminates. Further extending axially and outwardly from the neck 226 is the shank upper portion 208 that provides a connective or capture apparatus disposed at a distance from the upper end 232 and thus at a distance from a vertebra when the body 206 is implanted in such vertebra.

The shank upper portion 208 is configured for a fixed connection between the shank 204 and the retainer structure 212 and a pivotable connection between the shank 204/retainer structure 212 combination and the receiver 210 prior to fixing of the shank in a desired position with respect to the receiver 210. The upper portion 208 generally includes a substantially frusto-conical lower body 234 having a frusto-conical surface 235. The body 234 may include more than one frusto-conical surfaces graduating from the neck 226 to a convex, radially extending ring-like rib or ridge 238. The illustrated body 234 includes a lower frusto-conical surface 239 located near the neck 226 that is adjacent to the frusto-conical surface 235. The ridge 238 is sized and shaped to be received in a closely mating groove of the retainer 212, the rib and groove combination providing for secure engagement of the retainer 212 against the shank upper portion 208 at a desired location and orientation, prohibiting upward and downward movement of the retainer 212 along the shank axis AA. A curved, concave radially extending collar or flange 240 is located adjacent the rib 238 and extends outwardly from the axis AA to an outer surface 242 that is illustrated as semi-spherical in form, curving inwardly toward the axis AA in an upward direction toward a substantially planar annular upper surface 244, the surface 244 being disposed substantially perpendicular to the axis AA. The curved collar or flange 240 is sized and shaped to readily assemble with and closely receive a curved upper portion of the retainer 212 as will be described in greater detail below. An internal tool engagement drive feature or structure 246 is formed in the surface 244 and extends downwardly along the axis AA, substantially perpendicular to the upper surface 44 and is illustrated as a hex-shape structure sized and shaped to mate with hex driving tool (not shown) having an external drive configured to fit within the tool engagement structure 246 for both driving and rotating the shank body 206 into the vertebra. Although a hex-shaped drive 246 is illustrated, the drive 246 may have other shapes, including, but not limited to, a star-shaped form or other internal drive geometries. The drive 246 bottoms out at a planar surface 249, such surface also configured for engaging the driving tool. The shank 204 shown in the drawings is cannulated, having a small central bore 250 extending an entire length of the shank 204 along the axis AA. The bore 250 is defined by an inner cylindrical wall of the shank 204 and has a circular opening at the shank tip 228 and an upper opening communicating with the external drive 248 at the bottom surface 249. The bore 250 is coaxial with the threaded body 206 and the upper portion 208. The bore 250 provides a passage through the shank 204 interior for a length of wire (not shown) inserted into the vertebra prior to the insertion of the shank body 206, the wire providing a guide for insertion of the shank body 206 into the vertebra. To provide a biologically active interface with the bone, the threaded shank body 206 may be coated, perforated, made porous or otherwise treated as previously described herein with respect to the shank body 6 of the assembly 1.

With particular reference to FIGS. 17, 25 and 27-32, the receiver 110 has a generally U-shaped appearance with a partially discontinuous cylindrical inner profile (at the arms) and also a partially cylindrical, partially frusto-conical and partially spherical inner profile (at the base) as well as a partially curved and partially faceted outer profile. The receiver 110 has an axis of rotation BB that is shown in FIG. 17 as being aligned with and the same as the axis of rotation AA of the shank 204, such orientation being desirable during assembly of the receiver 210 with the shank 204 and the retainer 212. After the receiver 210 is pivotally attached to the shank 204, and the assembly 201 is implanted in the vertebra 213, the axis BB is typically disposed at an angle with respect to the axis AA, as shown, for example in FIGS. 30-32.

The receiver 210 includes a base 260 integral with a pair of opposed upstanding arms 262 forming a cradle and defining a channel 264 between the arms 262 with an upper opening, generally 266, and a lower seat 268, the channel 264 having a width for operably snugly receiving the rod 221 between the arms 262. Each of the arms 262 has an interior surface 270 that defines the inner cylindrical profile and includes a partial helically wound guide and advancement structure 272. In the illustrated embodiment, the guide and advancement structure 272 is a partial helically wound interlocking flangeform configured to mate under rotation with a similar structure on the closure structure 218, as described more fully below. However, it is foreseen that the guide and advancement structure 272 could alternatively be a square-shaped thread, a buttress thread, a reverse angle thread or other thread-like or non-thread-like helically wound discontinuous advancement structure for operably guiding under rotation and advancing the closure structure 18 downward between the arms 262, as well as eventual torquing when the closure structure 218 abuts against the rod 221 in some embodiments or abuts against the compression insert 214 in other embodiments.

At least one pair of opposed pair of tool receiving and engaging apertures 274 are formed on outer surfaces 276 of the arms 262. The illustrated embodiment further includes upper opposed apertures 273. The apertures 273 and 274 may be used for holding the receiver 210 during assembly with the shank 204 and the retainer structure 212, during the implantation of the shank body 206 into a vertebra (not shown) and assembly with the rod 221 and the closure structure 218. It is foreseen that tool receiving grooves or apertures may be configured in a variety of shapes and sizes and be disposed at other locations on the receiver arms 262. As illustrated, the apertures 274 do not extend completely through the arms 262. At each aperture 274, a thin wall 278 partially defines the aperture and may be crimped or pushed inwardly toward and into a cooperating aperture of the pressure insert 214 as will be described in greater detail below. Alternatively, the receiver 210 or the pressure insert 214 may be equipped with spring tabs that bias against a respective pressure insert or receiver to prohibit rotational movement of the insert 214 about the receiver axis BB once the insert 214 is loaded in the receiver 210 and positioned with the rod-receiving channel of the insert 214 in alignment with the U-shaped channel 264 of the receiver.

Communicating with and located beneath the channel 264 of the receiver 210 at the base portion 260 thereof is a chamber or cavity, generally 279, defined in part by an inner frusto-conical surface 280, an inner cylindrical surface 281 and a substantially spherical seating surface portion 282. The cavity 279 is also defined in part by a cylindrical inner wall 270′ located above and adjacent to the cylindrical surface 270, the wall 270′ being formed by the joining of the inner cylindrical walls 270 of each of the arms 262, the wall 270′ providing structure for the channel lower seat 268. The frusto-conical surface 280 is adjacent to and disposed between the cylindrical wall 270′ and the cylindrical wall 281 while the wall 281 is adjacent to the substantially spherical seating surface 282. It is noted that the surfaces 280 and 281 may also be radiused in some embodiments of the invention. The surface 282 is sized and shaped for slidably mating with the retainer structure 12 and ultimately frictionally mating therewith as will be described in greater detail below. The spherical surface portion 282 communicates with a lower opening neck 283 that communicates with both the cavity 279 and a receiver lower exterior or bottom 284 of the base 260. The neck 283 is substantially coaxially aligned with respect to the rotational axis BB of the receiver 210. The lower neck 283 is also sized and shaped to be smaller than an outer radial dimension of the retainer structure 212 when the retainer 212 is fixed to the shank upper portion 208, so as to form a restriction to prevent the structure 212 and attached shank portion 208 from passing through the cavity 279 and out the lower exterior 284 of the receiver 210 during operation thereof.

Furthermore, with particular reference to FIGS. 25-29, formed within each of the substantially cylindrical surfaces 270 of the arms 262 and located directly beneath the guide and advancement structure 272 is a recess 286 partially defined by a rounded stop or abutment wall 287. As will be described in greater detail below, the cooperating compression insert 214 includes a cooperating structure 288 that extends outwardly from each arm thereof that abuts against the respective abutment wall 287 of each of the receiver arms, providing a centering stop or block when the insert 214 is rotated into place in a clockwise manner as will be described below.

The retainer structure or retainer 212 is used to capture the shank upper portion 208 and retain the upper portion 208 within the receiver 210 as well as swivel or articulate with respect to the receiver 210. The retainer 212, best illustrated in FIGS. 17 and 21-24 has an operational central axis that is the same as the rotational axis AA associated with the shank 204, but when the retainer 212 is separated from the shank 204, the axis of rotation is identified as axis CC, as shown in FIG. 17. The retainer 212 has a central bore 291 that passes entirely through the retainer 212 from a top surface 292 to a bottom surface 294 thereof. Both the top surface 292 and the bottom surface 294 are substantially planar and disposed perpendicular to the axis CC. A first inner frusto-conical surface 296 defines a substantial portion of the bore 291, the surface 296 being adjacent to the bottom surface 294. The surface 296 is sized and shaped to be closely received about the shank surface 235 when the retainer 212 and the shank upper portion 208 are frictionally engaged within the receiver 210. A groove 298 extends radially outwardly from the axis CC and into the surface 296, the groove 298 being sized and shaped to closely receive the rib or ridge 238 of the shank upper portion 208. A convex radiused surface portion 300 extends between the groove 298 and the top surface 292. The outwardly curved surface portion 300 is sized and shaped to be closely received by and mate with the concave flanged collar 240 of the shank upper portion 208 during installation of the retainer 212 on the shank upper portion 208 within the receiver 210 as will be described in greater detail below.

The retainer 212 also has a radially outer partially spherically shaped surface 302 running between the top surface 292 and the bottom surface 294, the surface 302 being sized and shaped to mate with the partially spherical shaped seating surface 282 of the receiver 210. The surface 302 includes an outer radius that is larger than a radius of the neck lower opening 283 of the receiver 210 when the retainer 212 is in a neutral, non-compressed state, thereby prohibiting the retainer 212 and the shank upper portion 208 from passing through the neck 283 once the retainer 212 is fixed to the shank upper portion 208 within the receiver cavity 279. Although not required, it is foreseen that the outer partially spherically shaped surface 302 may be a high friction surface such as a knurled surface or the like.

As previously noted, the retainer 212 is an open ring and thus includes a gap formed by spaced end surfaces 304 and 305. In the illustrated embodiments, the surfaces 304 and 305 substantially face one another and are oriented at a slight angle with respect to one another, the surfaces 304 and 305 being slightly closer together at the outer surface 302 than at the inner frusto-conical surface 296. In other embodiments of the invention, the surfaces 304 and 305 may be parallel to one another. The illustrated surfaces 304 and 305 each run substantially parallel to the axis CC. In other embodiments, one or both surfaces may be at an obtuse angle with respect to the axis CC. The surfaces 304 and 305 are sized and shaped for allowing adequate clearance between the surfaces 304 and 305 when the retainer 212 is squeezed about the shank neck 226 and loaded with the shank upper portion 208 into the receiver 210 in a manner similar to that previously described with respect to the shank upper portion 8, the retainer 12 and the receiver 10 of the assembly 1. Once installed and locked into position, the retainer 212 closely grips the shank at the frusto-conical surface 235 and the rib 238, the surfaces 304 and 305 being in a substantially neutral, spaced position, with the inner frusto-conical surface 296 providing a substantially even and uniform gripping surface between the shank 204 and the receiver 210 at the spherical seating surface 282 when force is directed onto the shank domed surface 242 by the closure structure 218 pressing on the rod 221 that in turn presses on the compression insert 214. The frictionally mating radial rib 238 and groove 298 combination ensure a desired position and orientation of the retainer 212 with respect to the shank upper portion 208 regardless of other forces placed upon the retainer 212 within the receiver 210.

With particular reference to FIGS. 17 and 26-32, the compression insert 214 is sized and shaped to be received by and loaded into the receiver 210 as shown in FIG. 27. However, in other embodiments of the invention, the insert 214 may be sized for uploading or downloading into the receiver 210. The compression insert 214 has an operational central axis that is the same as the central axis BB of the receiver 210. The compression insert 214 has a central channel or through bore substantially defined by an inner cylindrical surface 320 coaxial with an inner partially spherical surface 322. The compression insert 214 through bore is sized and shaped to receive a driving tool (not shown) therethrough that engages the shank drive feature 246 when the shank body 206 is driven into bone. The surface 322 is sized and shaped to slidingly receive and ultimately frictionally engage the substantially spherical or domed surface 242 of the shank upper portion 208 such that the surface 322 initially slidingly and pivotally mates with the spherical surface 242. The surface 322 may include a roughening or surface finish to aid in frictional contact between the surface 322 and the surface 242, once a desired angle of articulation of the shank 204 with respect to the receiver 210 is reached.

The compression insert 214 also includes a pair of arms 324, each having a top surface 325, with a pair of U-shaped saddle-like surfaces 326 running between the arms and forming a seat for a longitudinal connecting member, such as the rod 21. Portions of the saddle surfaces 326 communicate with the bore defined by the cylindrical surface 320. The curved surfaces 326 are sized and shaped to closely receive the cylindrical rod 21 or other longitudinal connecting member. The saddle-like surfaces 326 extend between substantially planar opposed inner surfaces 328 of the arms 324, the inner surfaces 328 extending to the top surfaces 325 of the arms. The saddle-like surfaces 326 form a lower seat 330 located spaced from but near a lower or bottom surface 332 of the insert 214. The bottom surface 332 slopes upwardly from and communicates with the inner spherical surface 322, the surface 332 allowing for clearance between the insert 214 and the retainer 212 as best shown in FIG. 31. The insert arms 324 have a height dimension such that the top surfaces 325 are disposed above the rod 221 or other longitudinal connecting member captured by the assembly 201. The arms 324 preferably have an adequate thickness so that the arms 324 closely capture the rod 221 therebetween and also are supported by the cylindrical wall 270 defining the receiver arms located directly under the guide and advancement structure 272. In operation, the lower seat 330 (as well as at least a substantial portion of a remainder of the saddle 326) frictionally engages an outer surface 222 of the rod 221.

Formed in outer surface 335 of the arms 324 and located centrally with respect to each arm 324 is a shallow groove or depression 336. Each illustrated groove 336 is sized and shaped to cooperate with the apertures 274 and receiver thin inner walls 278 as will be described in greater detail below. The grooves 336 may be of any shape and are preferably elongate, running parallel to a central axis of the insert 214 that is operationally coaxial with the axis BB of the receiver 210. In some embodiments of the invention, the grooves or depressions 336 may be substantially flat surfaces formed by planing the cylindrical surface 335. The compression or pressure insert 214 ultimately seats on the shank upper portion 208 and is disposed substantially within the receiver inner surfaces 270 and 270′, with the thin walls 278 being pressed or crimped into each depression 336 to aid in holding the insert 214 in a desired alignment with respect to the rod 221 as will be described in greater detail below. In operation, the insert 214 extends at least partially in the channel 264 of the receiver 210 such that the saddle 326 surfaces substantially contact and engage the outer surface 222 of the rod 221 when such rod is placed in the receiver 210 and the closure structure or top 218 is tightened thereon. As will also be described below, the extending structure or stop feature 288 that is also located on each outer surface 335 of each insert arm 324 prohibits additional rotation of the insert 214 with respect to the receiver 210 during rotation and torquing of the closure top 218 against the rod 221 within the receiver arms 262.

With reference to FIGS. 17 and 30-32, the illustrated elongate rod or longitudinal connecting member 221 can be any of a variety of implants utilized in reconstructive spinal surgery, but is typically a cylindrical, elongate structure having the outer substantially smooth, cylindrical surface 222 of uniform diameter. The rod 221 may be made from a variety of metals, metal alloys and deformable and less compressible plastics, including, but not limited to rods made of elastomeric, polyetheretherketone (PEEK) and other types of materials. It is further noted that longitudinal connecting members for use with the assembly 201 may take a variety of shapes, including but not limited to rods or bars of oval, rectangular or other curved or polygonal cross-section. The shape of the insert 214 channel or saddle 326 may be modified so as to closely hold, and if desired, fix the longitudinal connecting member to the assembly 201. Some embodiments of the assembly 201 may also be used with a tensioned cord. Such a cord may be made from a variety of materials, including polyester or other plastic fibers, strands or threads, such as polyethylene-terephthalate. Furthermore, the longitudinal connector may be a component of a longer overall dynamic stabilization connecting member, with cylindrical or bar-shaped portions sized and shaped for being received by the compression insert 214 of the receiver having a u-shaped channel (or rectangular- or other-shaped channel) for closely receiving the longitudinal connecting member. The longitudinal connecting member may be integral or otherwise fixed to a bendable or damping component that is sized and shaped to be located between adjacent pairs of bone screw assemblies 201, for example. A damping component or bumper may be attached to the longitudinal connecting member at one or both sides of the bone screw assembly 201. A rod or bar (or rod or bar component) of a longitudinal connecting member may be made of a variety of materials ranging from deformable plastics to hard metals, depending upon the desired application. Thus, bars and rods of the invention may be made of materials including, but not limited to metal and metal alloys including but not limited to stainless steel, titanium, titanium alloys and cobalt chrome; or other suitable materials, including plastic polymers such as polyetheretherketone (PEEK), ultra-high-molecular weight-polyethylene (UHMWP), polyurethanes and composites, including composites containing carbon fiber, natural or synthetic elastomers such as polyisoprene (natural rubber), and synthetic polymers, copolymers, and thermoplastic elastomers, for example, polyurethane elastomers such as polycarbonate-urethane elastomers.

With reference to FIGS. 17 and 30-32, the closure structure or closure top 218 shown with the assembly 201 is rotatably received between the spaced arms 262 and is substantially similar to the closure 18 previously described herein with respect to the assembly 1 having a guide and advancement structure 362, a top surface 364, an internal drive 366, a bottom surface 368, a point 369 and a rim 370, the same or substantially similar to the respective guide and advancement structure 162, top surface 164, internal drive 166, bottom surface 168, point 169 and rim 170 of the closure 18.

It is noted that the closure 218 can be any of a variety of different types of closure structures for use in conjunction with the present invention with suitable mating structure on the upstanding arms 262. It is also foreseen that the closure top could be a twist-in or slide-in closure structure. It is also foreseen that the closure structure 218 may alternatively include a break-off head designed to allow such a head to break from a base of the closure at a preselected torque, for example, 70 to 140 inch pounds. Such a closure structure would also include a base having an internal drive to be used for closure removal. In some embodiments of the invention, the base 168 is planar and does not include a point or rim. In other embodiments, some or most of the base may be domed or radiused and may further include a surface feature, such as roughening for engagement with the surface 222 of the rod 221. The closure top 218 may further include a cannulation through bore (not shown) extending along a central axis thereof and through the top and bottom surfaces thereof. Such a through bore provides a passage through the closure 218 interior for a length of wire (not shown) inserted therein to provide a guide for insertion of the closure top into the receiver arms 262.

In other embodiments of the invention, the closure top 218 may include an annular base rim or step adjacent the bottom surface 368 sized and shaped such that such annular rim engages the top surfaces 325 of the insert 214 and presses the insert 214 down into pressing engagement with the shank upper portion 208 to lock the shank 204 in place with respect to the receiver 210. Thus, in some embodiments of the invention, the assembly 201 cooperates with a rod, cord, cable or other longitudinal connecting member to capture such connecting member within the receiver 210, but to allow the rod or other connector some freedom of movement within the receiver 210. In such applications, elastic spacers can be positioned around the connecting member and between the receivers. The closure 218 and insert 214 combination may also be desirable when the connecting member is made from a deformable plastic. In such embodiments, the closure bottom surface 368 may engage and frictionally hold the connecting member in place, but the polyaxial mechanism is firmly locked in place by the closure 218 directly engaging and pressing upon the insert 214 that in turn presses on the shank upper portion, desirably holding, but not over-stressing the longitudinal connecting member at the cite of engagement with the bone screw. Also, if a longitudinal connecting member would eventually become partially or totally disengaged from the closure bottom surface 368, for example, if a plastic connecting member exhibits creep, the shank 204 would advantageously remain fixed in position with respect to the receiver regardless of any movement of the connecting member within the receiver.

With reference to FIG. 25, prior to the polyaxial bone screw assembly 201 being placed in use according to the invention, the retainer 212 is loaded onto the shank 204 and the shank upper portion 208 and the squeezed or compressed retainer 212 are both bottom or uploaded into the receiver 210 at the neck 283 in a manner the same or substantially similar to the uploading of the retainer 12 and shank upper portion 8 of the assembly 1 previously described herein. The retainer 212 is then seated within the receiver 210 with the outer spherical surface 302 in sliding engagement with the receiver inner spherical seating surface 282. The shank upper portion 208 is then be pulled downwardly toward the base neck 283, with the resilient retainer 212 sliding upwardly along the shank frusto-conical surface 235. As the shank upper portion 208 moves downwardly, the curved retainer surface 300 contacts the rib 238 and is pushed radially outwardly, with the surface 300 sliding along the collar surface 240 of the shank upper portion 208 until the rib 238 is received into the groove 298. At this time, the retainer 212 resiliently moves or snaps into position about the rib 238 with the surface 296 also frictionally engaging the shank frusto-conical surface 235 (see FIG. 27).

With further reference to FIG. 27 and also to FIGS. 28 and 29, the compression insert 214 is then downloaded into the receiver 210 as indicated by the arrow L at the opening 266 with the arms 324 aligned in the channel 264 between the guide and advancement structures 272. The insert 214 is then moved downwardly in the channel and toward the cavity 279. Once the arms 324 are located generally below the guide and advancement structure 272, the insert 214 is rotated in a clock-wise direction about the axis BB of the receiver 210 and indicated by the arrow R. The arms 324 fit within the discontinuous cylindrical wall 270 of the receiver arms 262 and the structures 288 are received within the arm recesses 286. Once the arms 324 are located directly below the guide and advancement structures 272, further rotation is prohibited by the insert structures 288 each abutting against the abutment wall 287. A tool (not shown) is then used to press the thin walls 278 of the receiver 210 into the recesses or shallow grooves 336 of the insert 214 (see FIG. 29). The insert 214 is now locked into place with respect to rotation about the axis BB inside the receiver 210. Furthermore, although some upward and downward movement of the insert 214 is possible, the guide and advancement structures 272 prohibit upward movement of the insert 214 out of the channel 264. As illustrated in FIG. 28, the insert 214 seats on the shank upper portion surface 242 with the surface 322 in sliding engagement with the surface 242. At this time, the shank upper portion 208, the retainer structure 212, the receiver seating surface 282 and the lower aperture or neck 283 cooperate to maintain the shank body 206 in pivotal and rotational relation with the receiver 210. Only the retainer structure 212 is in slidable engagement with the receiver spherical seating surface 282. Both the shank upper portion 208 and the threaded portion of the shank body 206 are in spaced relation with the receiver 210. At this point there is no substantial outward or downward pressure on the shank upper portion 208 and so the retainer 212 is easily rotatable along with the shank 206 within the receiver chamber and such rotation is of a ball and socket type wherein the angle of rotation is only restricted by engagement of the shank neck 226 with the neck 283 of the receiver 210. The shank 204 is freely pivotable with respect to the receiver 210 until the insert 214 is pressed down upon the upper portion 208, placing the shank upper portion 208 into locking frictional engagement with the receiver 210 at the surface 282.

The bone screw assembly 201 made up of the assembled shank 204, receiver 210, retainer 212 and insert 214 is then normally screwed into a bone, such as the vertebra 213, by rotation of the shank 204 using a suitable driving tool (not shown) that operably drives and rotates the shank body 206 by engagement thereof at the internal drive 246. Specifically, the vertebra may be pre-drilled to minimize stressing the bone and have a guide wire (not shown) inserted to provide a guide for the placement and angle of the shank 204 with respect to the vertebra. A further tap hole may be made using a tap with the guide wire as a guide. Then, the bone screw assembly is threaded onto the guide wire utilizing the cannulation bore 250 by first threading the wire into the opening at the bottom 228 and then out of the top opening at the drive feature 246. The shank 204 is then driven into the vertebra using the wire as a placement guide. It is foreseen that the bone screw assembly 201, the rod 221 (also having a central lumen in some embodiments) and the closure top 218 (also with a central bore) can be inserted in a percutaneous or minimally invasive surgical manner, utilizing guide wires.

With reference to FIGS. 30-32, the rod 221 is eventually positioned in an open or percutaneous manner in cooperation with the at least two bone screw assemblies 201. Alignment of the rod surface 222 with the saddle 326 of the insert 214 is initially provided and then maintained by the engagement between the insert structures 288 abutting against the receiver walls 287 as well as the crimped walls 278 of the receiver 210 pressing into the insert grooves 336. The closure structure 218 is then inserted into and advanced between the arms 262 of each of the receivers 210. Each closure structure 18 is rotated, using a tool engaged with the inner drive 366 until a selected torque is reached at which point the rod 221 engages the saddle 326 and the rod is urged toward, but not in contact with the lower seat of the receiver 210 that defines the U-shaped channel 264. For example, about 80 to about 120 inch pounds pressure may be required for fixing the bone screw shank 206 with respect to the receiver 210.

As the closure structure 218 rotates and moves downwardly into the respective receiver 210, the point 369 and rim 370 engage and penetrate the rod surface 222, the closure structure 218 pressing against and biasing the rod 221 into engagement with the compression insert 214 that operably produces a frictional engagement between the insert surface 322 and the shank surface 242 and also urges the shank upper portion 208 toward the retainer 212 and, in turn, the structure 212 in a direction toward the base 260 of the receiver 210, so as to frictionally seat the retainer spherical surface 302 against the internal spherical seating surface 282 of the receiver 210, also fixing the shank 204 and the retainer 212 in a selected, rigid position relative to the receiver 210. At this time it is also possible for the retainer 212 to expand somewhat for an even tighter fit in the receiver cavity lower seat 282.

If removal of the rod 221 from any of the bone screw assemblies 201 is necessary, or if it is desired to release the rod 221 at a particular location, disassembly is accomplished by using the driving tool (not shown) that mates with the internal drive 366 on the closure structure 218 to rotate and remove such closure structure from the cooperating receiver 210. Disassembly is then accomplished in reverse order to the procedure described previously herein for assembly.

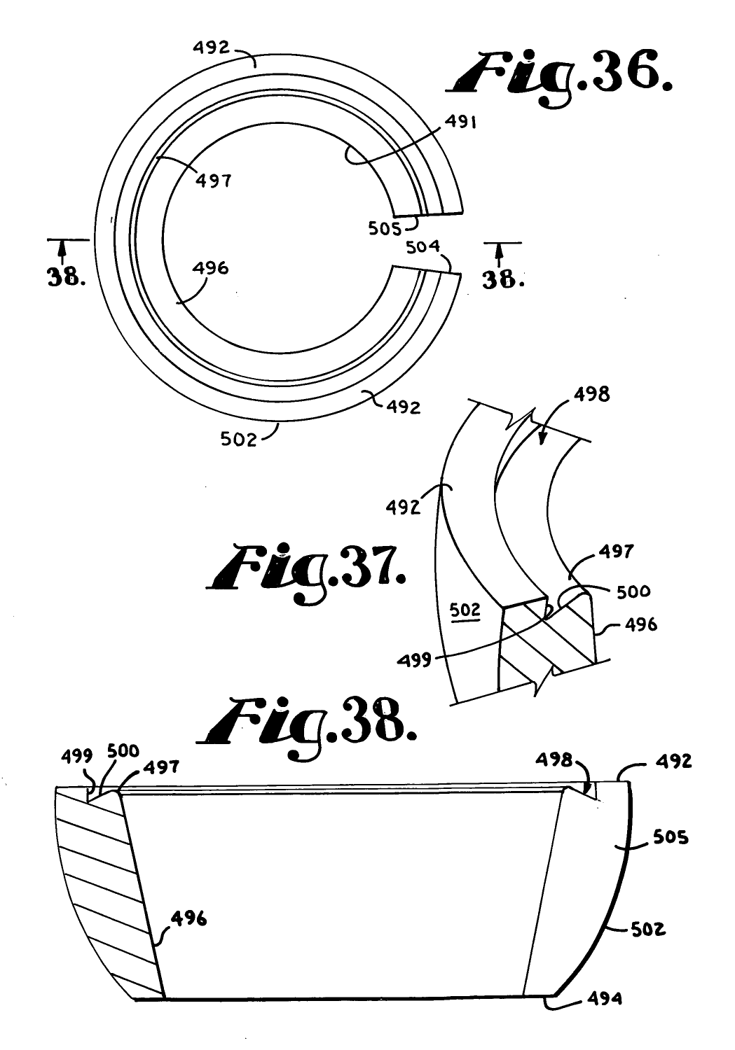

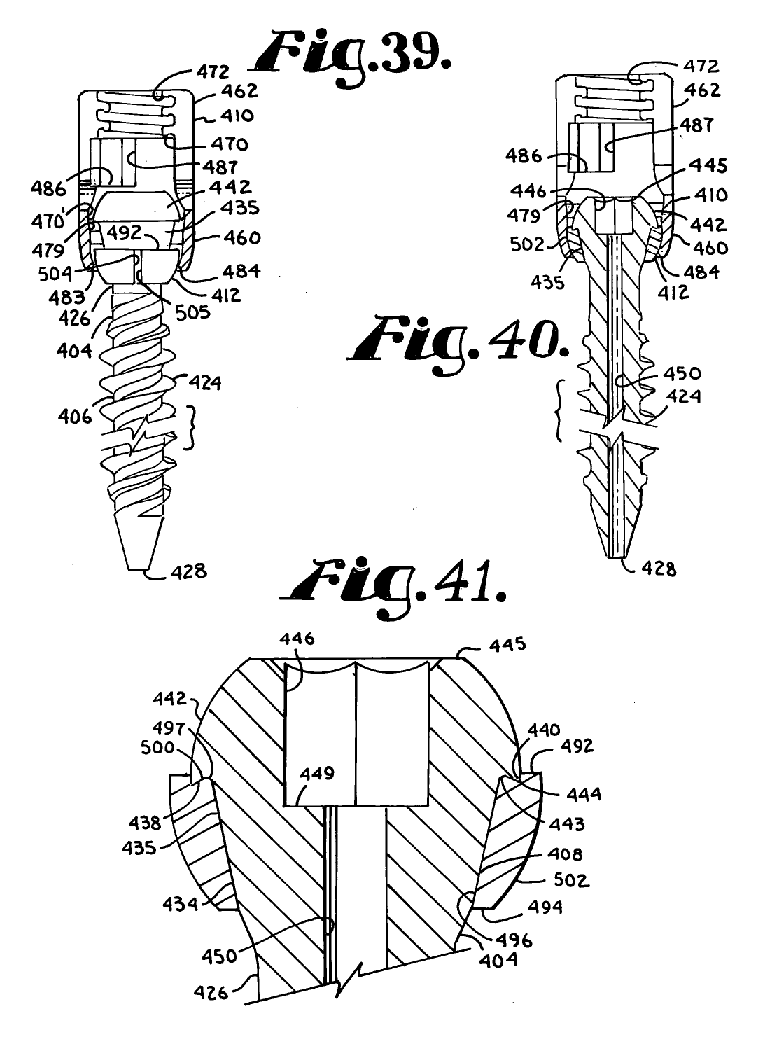

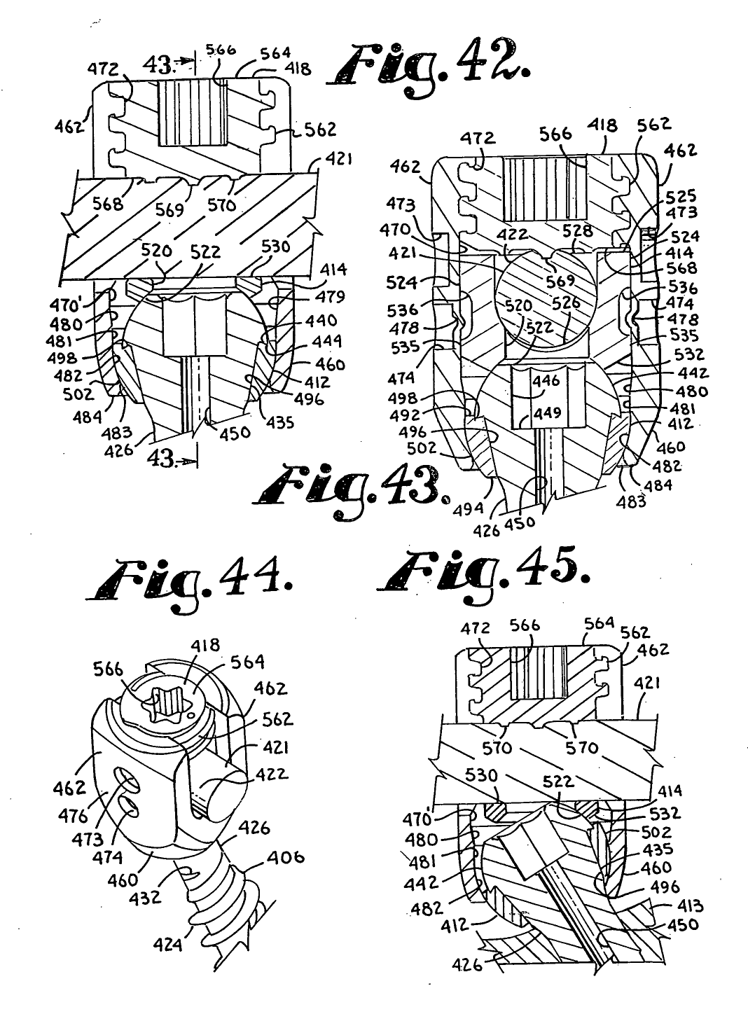

With reference to FIGS. 33-45, a third embodiment of a polyaxial bone screw assembly according to the invention, generally 401, includes a shank 404 having a body 406 and an upper portion 408, a receiver 410, a retainer 412, a compression insert 414 and a closure structure 418 and is shown with a longitudinal connecting member in the form of a hard, inelastic, substantially non-deformable rod 421 having a substantially cylindrical outer surface 422. The assembly 401 is substantially similar to the assembly 201 with the exception of certain features of the radial locking interface between the shank upper portion and the retainer.

The shank 404, best illustrated in FIGS. 33-35 and 41, is elongate, with the shank body 406 having a helically wound bone implantable thread 424 (single or dual lead thread form) extending from near a neck 426 located adjacent to the upper portion or capture structure 408, to a tip 428 of the body 406 and extending radially outwardly therefrom. During use, the body 406 utilizing the thread 424 for gripping and advancement is implanted into a vertebra 413 leading with the tip 428 and driven down into the vertebra with an installation or driving tool (not shown), so as to be implanted in the vertebra to near the neck 426. The shank 404 has an elongate axis of rotation generally identified by the reference letter AAA.

The neck 406 extends axially upward from the shank body 406. The neck 426 may be of the same or slightly reduced radius as compared to an adjacent upper end or top 432 of the body 406 where the thread 424 terminates. Further extending axially and outwardly from the neck 426 is the shank upper portion 408 that provides a connective or capture apparatus disposed at a distance from the upper end 432 and thus at a distance from a vertebra when the body 406 is implanted in such vertebra.

The shank upper portion 408 is configured for a fixed connection between the shank 404 and the retainer structure 412 and a pivotable connection between the shank 404/retainer structure 412 combination and the receiver 410 prior to fixing of the shank in a desired position with respect to the receiver 410. The upper portion 408 generally includes a substantially frusto-conical lower body 434 having a frusto-conical surface 435. The body 434 may include more than one frusto-conical surface graduating from the neck 426 to near a surface 438 defining an undercut in a lower peripheral outer portion 440 of a domed or partially spherical surface 442 sized and shaped to slidingly mate with a concave spherical surface of the insert 414 described in greater detail below. With particular reference to FIG. 41, the surface 438 is disposed at an acute angle with respect to the frusto-conical surface 435. Thus the outer portion 440 is an overhanging portion created in part by the surface 438. In the illustrated embodiment, a narrow annular strip or surface 443 is adjacent to and disposed between the frusto-conical surface 435 and the surface 438, providing a narrow separation between such surfaces 435 and 438 and increasing the undercut area below the portion 440 of the domed surface 442. Thus, the surface 438 defining the undercut runs from the strip or surface 443 radially outwardly and downwardly in a direction toward the shank tip 428 to a lower and outer edge 444 of the convex radiused surface 442. The overhanging portion 440 and particularly the edge 444 is configured for being received in a recessed or grooved surface of the retainer 412 as will be described in greater detail below, prohibiting upward movement of the retainer 412 along the shank axis AAA. In a top surface 445, an internal tool engagement drive feature or structure 446 is formed that extends downwardly along the axis AAA, substantially perpendicular to the upper surface 445 and is illustrated as a hex-shape structure sized and shaped to mate with hex driving tool (not shown) having an external drive configured to fit within the tool engagement structure 446 for both driving and rotating the shank body 406 into the vertebra. Although a hex-shaped drive 446 is illustrated, the drive 446 may have other shapes, including, but not limited to, a star-shaped form or other internal drive geometries. The drive 446 bottoms out at a planar surface 449, such surface also configured for engaging the driving tool. The shank 404 shown in the drawings is cannulated, having a small central bore 450 extending an entire length of the shank 404 along the axis AAA. The bore 450 is defined by an inner cylindrical wall of the shank 404 and has a circular opening at the shank tip 428 and an upper opening communicating with the external drive 448 at the bottom surface 449. The bore 450 is coaxial with the threaded body 406 and the upper portion 408. The bore 450 provides a passage through the shank 404 interior for a length of wire (not shown) inserted into the vertebra prior to the insertion of the shank body 406, the wire providing a guide for insertion of the shank body 406 into the vertebra. To provide a biologically active interface with the bone, the threaded shank body 406 may be coated, perforated, made porous or otherwise treated as previously described herein with respect to the shank body 6 of the assembly 1.

With particular reference to FIGS. 33 and 42-45, the receiver 410 is identical or substantially similar to the receiver 210 previously described herein with respect to the assembly 201. Therefore, the receiver 410 includes the features of: an axis BBB; a base 460; opposed arms 462; a channel 464 with an upper opening 466 and a lower seat 468; an arm inner surface 470 with a guide and advancement structure 472 and an inner surface 470′ extending from the arm surfaces 470 into and about the base 460; upper 473 and lower 474 tool engaging apertures on outer arm surfaces 476, a thin wall 478 partially defining each lower tool engaging aperture 474; a receiver chamber or cavity 470 defined in part by a frusto-conical surface 480, a cylindrical surface 481, a spherical seating surface 482 and a neck 483 opening to a lower exterior surface 484; and a recess 486 disposed in each arm inner surface 470 defined in part by a rounded stop or abutment wall 487 that cooperates with a structure or stop 488 on the cooperating compression insert 414; such features being the same or substantially similar in form and function to the respective assembly 201 features of: the axis BB; the base 260; opposed arms 262; the channel 264 with the upper opening 266 and a lower seat 268; the arm inner surface 270 having the guide and advancement structure 272, and the inner surface 270′ extending from the arm surfaces 270 into and about the base 260; upper 273 and lower 274 tool engaging apertures on each of the outer arm surfaces 276 and the thin wall 278 partially defining each lower tool engaging aperture 274; the receiver chamber or cavity 270 defined at least in part by the frusto-conical surface 280, the cylindrical surface 281, the spherical seating surface 282 and the neck 283 opening to the lower exterior surface 284; and the recess 286 disposed in each arm inner surface 270 defined in part by the rounded stop or abutment wall 287 that cooperates with the structure or stop 288 on the cooperating compression insert 214 of the receiver 210, all such features being previously described herein with respect to the assembly 201.

The retainer structure or retainer 412 is used to capture the shank upper portion 408 and retain the upper portion 408 within the receiver 410 as well as swivel or articulate with respect to the receiver 410. The retainer 412, best illustrated in FIGS. 33 and 36-41 has an operational central axis that is the same as the rotational axis AAA associated with the shank 404, but when the retainer 412 is separated from the shank 404, the axis of rotation is identified as axis CCC, as shown in FIG. 33. The retainer 412 has a central bore, generally 491, that passes entirely through the retainer 412 from a top surface 492 to a bottom surface 494 thereof. Both the illustrated top surface 492 and bottom surface 494 are substantially planar and disposed perpendicular to the axis CCC. An inner frusto-conical surface 496 defines a substantial portion of the bore 491, the surface 496 being adjacent to the bottom surface 494 and extending upwardly to an annular rounded inner rim 497. The surface 496 is sized and shaped to be closely received about the shank surface 435 when the retainer 412 and the shank upper portion 408 are frictionally engaged within the receiver 410. Formed in the top surface 492 and extending inwardly to the rounded rim 497 is an annular groove or cut-out, generally 498 further defined by a radiused or partially spherical surface 499 and a sloping surface 500. The radiused surface 499 cuts centrally into the upper surface 492, running substantially perpendicular thereto while the surface 500 runs between the surface 499 and the rim 497, the rim being disposed slightly lower than the surface 492 with respect to the bottom surface 496. The groove or cut-out 498 is sized and shaped to fully receive the outer overhanging portion 440 of the shank surface 442. Specifically, the radiused surface 499 is sized and shaped to receive and surround the surface 442 of the shank upper portion 408 located near the edge 444; the surface 400 is sized and shaped to engage the undercut surface 438 of the shank top portion 408; and the rounded inner rim 497 is sized and shaped to engage the lower surface 443 that also defines the undercut that forms the overhanging portion 440 of the shank upper portion 408. The fixed radial relationship or locking provided by the shank overhanging portion 440 engaging the retainer groove 498 operatively functions to prohibit the retainer 412 from moving too far upwardly along the shank frusto-conical surface 435. However, the retainer 412 remains at a desirable spaced distance from the compression insert 414 during operation of the assembly 401 in any and all articulations of the shank 404 with respect to the receiver 410.

The retainer 412 also has a radially outer partially spherically shaped surface 502 running between the top surface 492 and the bottom surface 494, the surface 502 being sized and shaped to mate with the partially spherical shaped seating surface 482 of the receiver 410. The surface 502 includes an outer radius that is larger than a radius of the neck lower opening 483 of the receiver 410 when the retainer 412 is in a neutral, non-compressed state, thereby prohibiting the retainer 412 and the shank upper portion 408 from passing through the neck 483 once the retainer 412 is fixed to the shank upper portion 408 within the receiver cavity 479. Although not required, it is foreseen that the outer partially spherically shaped surface 502 may be a high friction surface such as a knurled surface or the like.

As previously noted, the retainer 412 is an open ring and thus includes a gap formed by spaced end surfaces 504 and 505. In the illustrated embodiments, the surfaces 504 and 505 substantially face one another and are oriented at a slight angle with respect to one another, the surfaces 504 and 505 being slightly closer together at the inner surface 496 than at the outer spherical surface 502. In other embodiments of the invention, the surfaces 504 and 505 may be parallel to one another. The illustrated surfaces 504 and 505 each run substantially parallel to the axis CCC. In other embodiments, one or both surfaces may be at an obtuse angle with respect to the axis CCC. The surfaces 504 and 505 are sized and shaped for allowing adequate clearance between the surfaces 504 and 505 when the retainer 512 is squeezed about the shank neck 526 and loaded with the shank upper portion 508 into the receiver 510 in a manner similar to that previously described with respect to the shank upper portion 8, the retainer 12 and the receiver 10 of the assembly 1. Once installed and locked into position, the retainer 512 closely grips the shank at the frusto-conical surface 435 and the shank over-hanging portion 440, the surfaces 504 and 505 being in a substantially neutral, spaced position, with the inner frusto-conical surface 496 providing a substantially even and uniform gripping surface between the shank 404 and the receiver 410 at the spherical seating surface 482 when force is directed onto the shank domed surface 442 by the closure structure 418 pressing on the rod 421 that in turn presses on the compression insert 414. The frictionally mating overhang 440 and groove retainer groove 498 combination ensure a desired position and orientation of the retainer 412 with respect to the shank upper portion 408 regardless of other forces placed upon the retainer 412 within the receiver 410.

It is foreseen that in other embodiments according to the invention, other radial locking combinations may be provided for use with the frusto-conical interface between the shank upper portion and the retainer. It is foreseen, for example, that the retainer groove 498 may be omitted with the retainer top surface directly abutting against a horizontal radially extending surface defining an overhang or radially extending portion of the shank upper spherical surface 442 and such a combination may further include a lower radial lip on the shank frusto-conical body 434 for engaging the retainer bottom surface 494 and thus capturing the retainer 212 between such a lip and the shank upper spherical surface 442 to prohibit axial movement of the retainer 412 along the shank axis BBB.

With particular reference to FIGS. 33 and 42-45, the compression insert 414 is identical or substantially similar in form and function to the insert 214 previously described herein with respect to the assembly 201. Thus, the insert 213 includes an inner cylindrical surface 520, an inner spherical surface 522, opposed arms 524 each having a top surface 525, saddle surfaces 526, planar inner surfaces 528, a lower seat 530, a bottom surface 532, outer arm surfaces 535 with a shallow groove 536, and the stop structure 488 also located on the outer arms the same or substantially similar to the respective inner cylindrical surface 320, inner spherical surface 322, opposed arms 324 each having a top surface 325, saddle surfaces 326, planar inner surfaces 328, lower seat 330, bottom surface 332, outer arm surfaces 335, shallow groove 336 and stop structure 288 previously described herein with respect to the insert 214 of the assembly 201.

With reference to FIGS. 33 and 42-45, the illustrated elongate rod or longitudinal connecting member 421 can be any of a variety of implants utilized in reconstructive spinal surgery, but is typically a cylindrical, elongate structure having the outer substantially smooth, cylindrical surface 422 of uniform diameter. The illustrated rod 421 is the same or substantially similar to the rods 221 and 21 previously described herein and may be made from a variety of materials as previously described herein with respect to the rods 221 and 21.

With reference to FIGS. 33 and 42-45, the closure structure or closure top 418 shown with the assembly 401 is the same or substantially similar to the closure top 218 previously described herein with respect to the assembly 201. Thus, the closure top 418 having a guide and advancement structure 562, a top surface 564, an internal drive 566, a bottom surface 568, a point 569 and a rim 570, the same or substantially similar in form and function to the respective guide and advancement structure 362, top surface 364, internal drive 366, bottom surface 368, point 369 and rim 370 of the closure 218 previously described herein with respect to the assembly 201. It is noted that the closure 418 can be any of a variety of different types of closure structures with different features for cooperating with a variety of longitudinal connecting members of different shapes and materials with suitable mating structure on the upstanding arms 462 as also previously described herein with respect to the closure top 218.

With reference to FIG. 39, prior to the polyaxial bone screw assembly 401 being placed in use according to the invention, the retainer 412 is loaded onto the shank 404 beneath the shank upper portion 408 and then the squeezed or compressed retainer 412 and upper portion 408 are both bottom or uploaded into the receiver 410 at the neck 483 in a manner the same or substantially similar to the uploading of the retainer 12 and shank upper portion 8 of the assembly 1 previously described herein. The retainer 412 is then seated within the receiver 410 with the outer spherical surface 502 in sliding engagement with the receiver inner spherical seating surface 482. The shank upper portion 408 is then be pulled downwardly toward the base neck 483, with the retainer 412 sliding upwardly along the shank frusto-conical surface 435. As the shank upper portion 408 moves downwardly, frusto-conical surfaces 435 and 496 slidingly engage until the overhanging portion 440 is received in the retainer groove 498 as best shown in FIGS. 40 and 41, the retainer 412 substantially surrounding a portion of the shank upper spherical surface 442, the undercut surface 438 firmly abutting against the sloping surface 500 of the retainer 412 and the retainer inner rim 497 abutting the shank undercut lower surface 443. At this point there is no substantial outward or downward pressure on the shank upper portion 408 and so the retainer 412 is easily rotatable along with the shank 406 within the receiver chamber and such rotation is of a ball and socket type wherein the angle of rotation is only restricted by engagement of the shank neck 426 with the neck 483 of the receiver 410. The shank 404 is freely pivotable with respect to the receiver 410 until the insert 414 is pressed down upon the upper portion 408, placing the shank upper portion 408 into locking frictional engagement with the receiver 410 at the surface 482.

With reference to FIGS. 42-45, the compression insert 414, rod 421 and closure top 418 are loaded into the receiver 410 in a manner the same or substantially similar as previously described herein with respect to the insert 214, rod 221 and closure top 218 of the assembly 201.

If removal of the rod 421 from any bone screw assemblies 401 is necessary, or if it is desired to release the rod 421 at a particular location, disassembly is accomplished by using the driving tool (not shown) that mates with the internal drive 566 on the closure structure 418 to rotate and remove such closure structure from the cooperating receiver 410. Disassembly is then accomplished in reverse order to the procedure described previously herein for assembly.

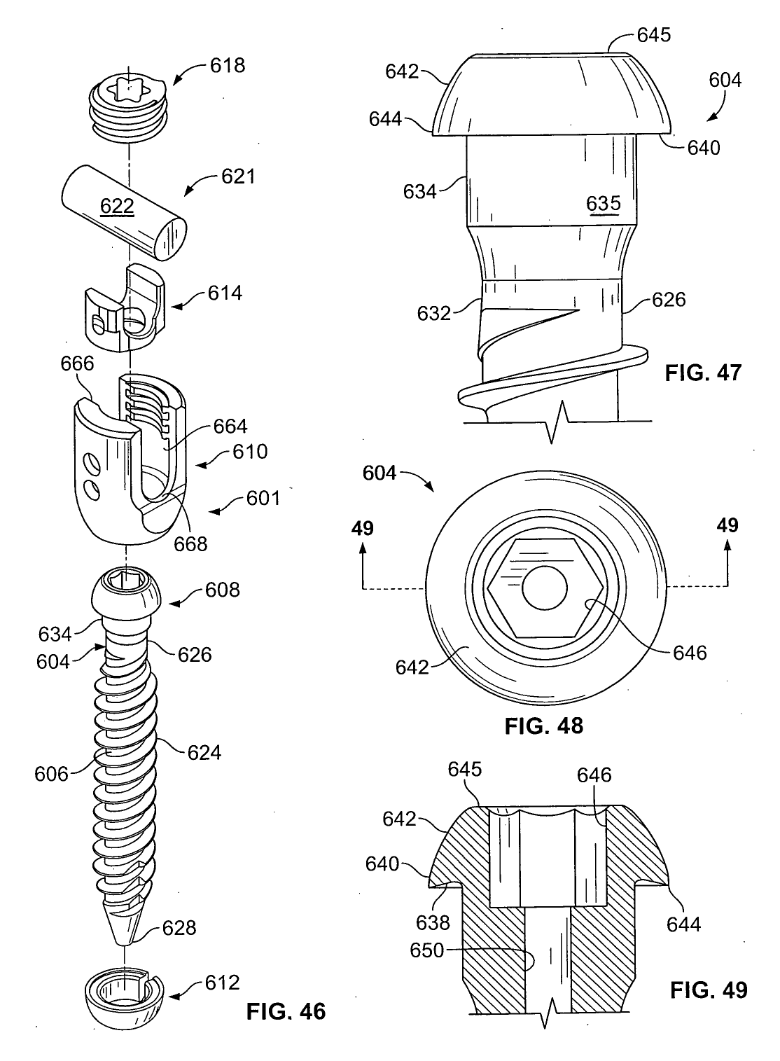

With reference to FIGS. 46-54, a fourth embodiment of a polyaxial bone screw assembly according to the invention, generally 601, includes a shank 604 having a body 606 and an upper portion or capture structure 608, a receiver 610, a retainer 612, a compression insert 614 and a closure structure 618 and is shown with a longitudinal connecting member in the form of a hard, inelastic, substantially non-deformable rod 621 having a substantially cylindrical outer surface 622. The assembly 601 is substantially similar to the assembly 401 with the exception of certain features of the radial locking interface between the shank upper portion and the retainer. More specifically, a lower body 634 of the upper portion 608 is cylindrical instead of frusto-conical.

The shank 604, best illustrated in FIGS. 46-49, is elongate, with the shank body 606 having a helically wound bone implantable thread 624 (single or dual lead thread form) extending from near a neck 626 located adjacent to the upper portion or capture structure 608, to a tip 628 of the body 606 and extending radially outwardly therefrom.

The neck 606 extends axially upward from the shank body 606. The neck 626 may be of the same or slightly reduced radius as compared to an adjacent upper end or top 632 of the body 606 where the thread 624 terminates. Further extending axially and outwardly from the neck 626 is the shank capture structure 608 that provides a connective or capture apparatus disposed at a distance from the upper end 632 and thus at a distance from a vertebra when the body 606 is implanted in such vertebra.

The shank capture structure 608 is configured for a fixed connection between the shank 604 and the retainer 612 and a pivotable connection between the shank 604/retainer 612 combination and the receiver 610 prior to fixing of the shank in a desired position with respect to the receiver 610. The capture structure 608 generally includes a substantially cylindrical lower body 634 having a cylindrical surface 635. The cylindrical lower body 634 of capture structure 608 extends from the neck 626 to near a surface 638 defining an undercut in a lower peripheral outer portion 640 of a domed or partially spherical surface 642 sized and shaped to slidingly mate with a concave spherical surface of the insert 614 described in greater detail below. With particular reference to FIG. 49, the surface 638 is disposed at an acute angle with respect to the cylindrical surface 635. Thus the outer portion 640 is an overhanging portion created in part by the surface 638. The undercut surface 638 slopes downward from its intersection with cylindrical surface 635 outward toward the lower peripheral outer portion 640 of domed surface 642.

The overhanging portion 640 and particularly lower, outer edge 644 is configured for being received in a recessed or grooved surface of the retainer 612 as will be described in greater detail below, prohibiting upward movement of the retainer 612 along the shank longitudinal axis. In a top surface 645, an internal tool engagement drive feature or structure 646 is formed that extends downwardly along the shank longitudinal axis, substantially perpendicular to the upper surface 645 and is illustrated as a hex-shape structure sized and shaped to mate with hex driving tool (not shown) having an external drive configured to fit within the tool engagement structure 646 for both driving and rotating the shank body 606 into the vertebra. Although a hex-shaped drive 646 is illustrated, the drive 646 may have other shapes, including, but not limited to, a star-shaped form or other internal drive geometries. The shank 604 shown in the drawings is cannulated, having a small central bore 650 extending an entire length of the shank 604 along its longitudinal axis.

Referring to FIGS. 46, 53 and 54, the receiver 610 is identical or substantially similar to the receiver 410 previously described herein with respect to the assembly 401. The receiver 610 includes the features of: a base 660; opposed arms 662; a channel 664 with an upper opening 666 and a lower seat 668; a receiver chamber or cavity 670 defined in part by a spherical seating surface 682 and a neck 683 opening to a lower exterior surface 684.

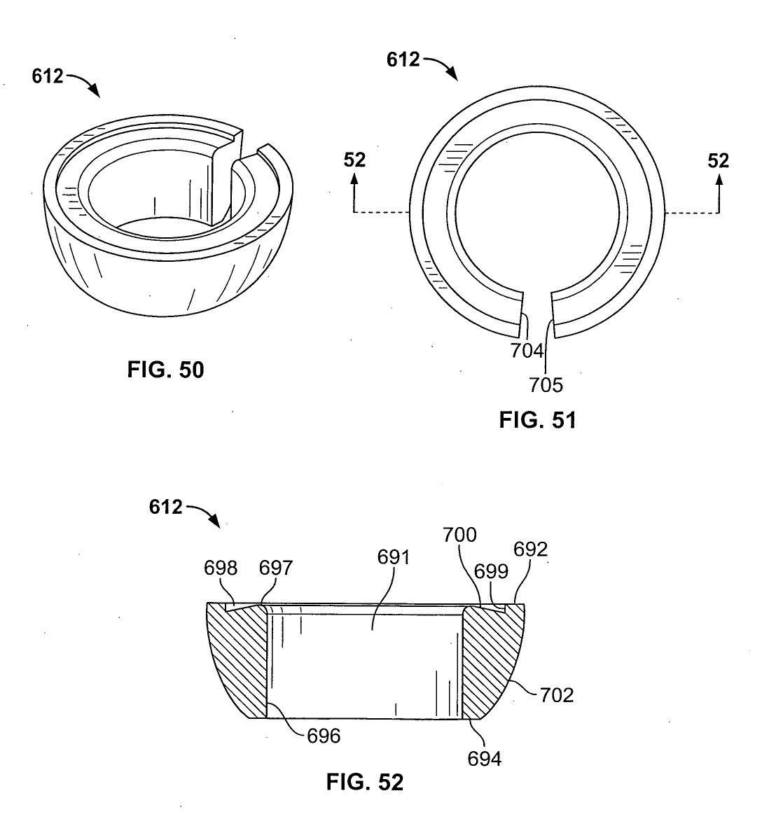

The retainer structure or retainer 612 is used to capture the shank capture structure 608 and retain the capture structure 608 within the receiver 610 as well as swivel or articulate with respect to the receiver 610. The retainer 612, best illustrated in FIGS. 50-52, has a central bore, generally 691, that passes entirely through the retainer 612 from a top surface 692 to a bottom surface 694 thereof. An inner cylindrical surface 696 defines a substantial portion of the bore 691, the surface 696 being adjacent to the bottom surface 694 and extending upwardly to an annular rounded inner rim 697. The surface 696 is sized and shaped to be closely received about the shank cylindrical surface 635 when the retainer 612 and the shank capture structure 608 are frictionally engaged within the receiver 610.

Formed in the top surface 692 and extending inwardly to the rounded rim 697 is an annular groove or cut-out, generally 698 further defined by a radiused or partially spherical surface 699 and a sloping surface 700. The radiused surface 699 cuts centrally into the upper surface 692, running substantially perpendicular thereto while the surface 700 runs between the surface 699 and the rim 697, the rim being disposed slightly lower than the top surface 692. The groove or cut-out 698 is sized and shaped to fully receive the outer overhanging portion 640 of the shank surface 642. Specifically, the radiused surface 699 is sized and shaped to receive and surround the surface 642 of the shank upper portion 608 located near the edge 644; and the surface 700 is sized and shaped to engage the undercut surface 638 of the shank top portion 608. The fixed radial relationship or locking provided by the shank overhanging portion 640 engaging the retainer groove 698 operatively functions to prohibit the retainer 612 from moving upward past the domed portion 642 of the shank head 608. However, the retainer 612 remains at a desirable spaced distance from the compression insert 614 during operation of the assembly 601 in any and all articulations of the shank 604 with respect to the receiver 610.

The retainer 612 also has a radially outer partially spherically shaped surface 702 running between the top surface 692 and the bottom surface 694, the spherical surface 702 being sized and shaped to mate with the partially spherical shaped seating surface 682 of the receiver 610. The surface 702 includes an outer radius that is larger than a radius of the neck lower opening 683 of the receiver 610 when the retainer 612 is in a neutral, non-compressed state, thereby prohibiting the retainer 612 and the shank upper portion 608 from passing through the neck 683 once the retainer 612 is fixed to the shank upper portion 608 within the receiver chamber 670. Although not required, it is foreseen that the outer partially spherically shaped surface 702 may be a high friction surface such as a knurled surface or the like.

The retainer 612 is an open ring and thus includes a gap formed by spaced end surfaces 704 and 705. In the illustrated embodiments, the surfaces 704 and 705 substantially face one another and are oriented at a slight angle with respect to one another, the surfaces 704 and 705 being slightly closer together at the inner surface 696 than at the outer spherical surface 702. In other embodiments of the invention, the surfaces 704 and 705 may be parallel to one another or closer together at the outer spherical surface 702 than the inner surface 696.

The surfaces 704 and 705 are sized and shaped for allowing adequate clearance between the surfaces 704 and 705 when the retainer 612 is squeezed about the shank neck 626 and loaded with the shank capture structure 608 into the receiver 610 in a manner similar to that previously described with respect to the shank upper portion 8, the retainer 12 and the receiver 10 of the assembly 1.

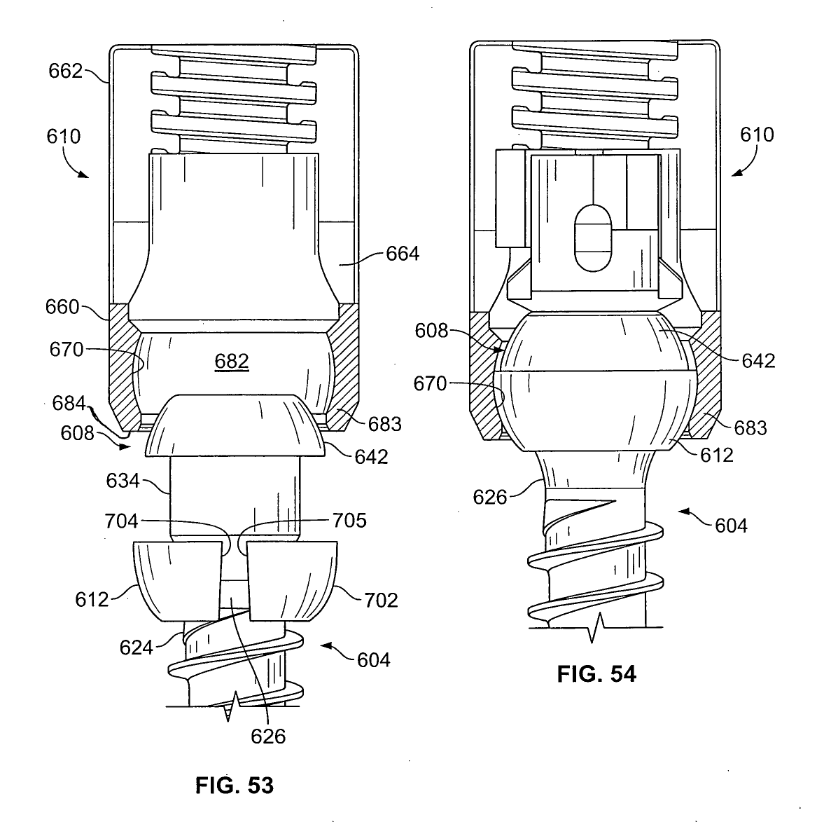

With reference to FIG. 53, prior to the polyaxial bone screw assembly 601 being placed in use according to the invention, the retainer 612 is loaded onto the shank 604 around the neck 626 and below the shank upper portion or capture structure 608 and then the squeezed or compressed to reduce the outer diameter of the retainer 612 to a diameter smaller than the diameter of the opening extending through the neck 683 of the receiver 610. The retainer 612 and capture structure 608 of shank 604 are both bottom or uploaded into the receiver 610 at the neck 683 in a manner substantially similar to the uploading of the retainer 12 and shank upper portion 8 of the assembly 1 previously described herein.

The retainer 612 is then allowed to expand and seated within the receiver 610 with the outer spherical surface 702 in sliding engagement with the receiver inner spherical seating surface 682. The shank upper portion 608 is then pulled downwardly toward the base neck 683, with the retainer 612 sliding upwardly along the shank cylindrical surface 635. As the shank upper portion 608 moves downwardly, cylindrical surfaces 635 and 696 slidingly engage until the overhanging portion 640 is received in the retainer groove 698 as best shown in FIG. 54, the retainer 612 substantially surrounding a portion of the shank upper spherical surface 642, and the undercut surface 638 firmly abutting against the sloping surface 700 of the retainer 612. At this point there is no substantial outward or downward pressure on the shank upper portion 608 and so the retainer 612 is easily rotatable along with the shank 604 within the receiver chamber and such rotation is of a ball and socket type wherein the angle of rotation is only restricted by engagement of the shank neck 626 with the neck 683 of the receiver 610. The shank 604 is freely pivotable with respect to the receiver 610 until the insert 614 is pressed down upon the upper portion 608, placing the shank upper portion 608 into locking frictional engagement with the receiver 610 at the surface 682.

With reference to FIG. 54, the compression insert 614, rod 621 and closure top 618 are loaded into the receiver 610 in a manner the same or substantially similar as previously described herein with respect to the insert 414, rod 421 and closure top 418 of the assembly 401.

Once installed and locked into position, the retainer 612 closely grips the shank at the cylindrical surface 635 and the shank over-hanging portion 640, the surfaces 704 and 705 being in a substantially neutral, spaced position, with the inner cylindrical surface 696 of the retainer providing a substantially even and uniform gripping surface between the shank 604 and the receiver 610 at the spherical seating surface 682 when force is directed onto the shank domed surface 642 by the closure structure 618 pressing on the rod 621 that in turn presses on the compression insert 614. The frictionally mating overhang 640 and retainer groove 698 combination ensure a desired position and orientation of the retainer 612 with respect to the shank upper portion 608 regardless of other forces placed upon the retainer 612 within the receiver 610.

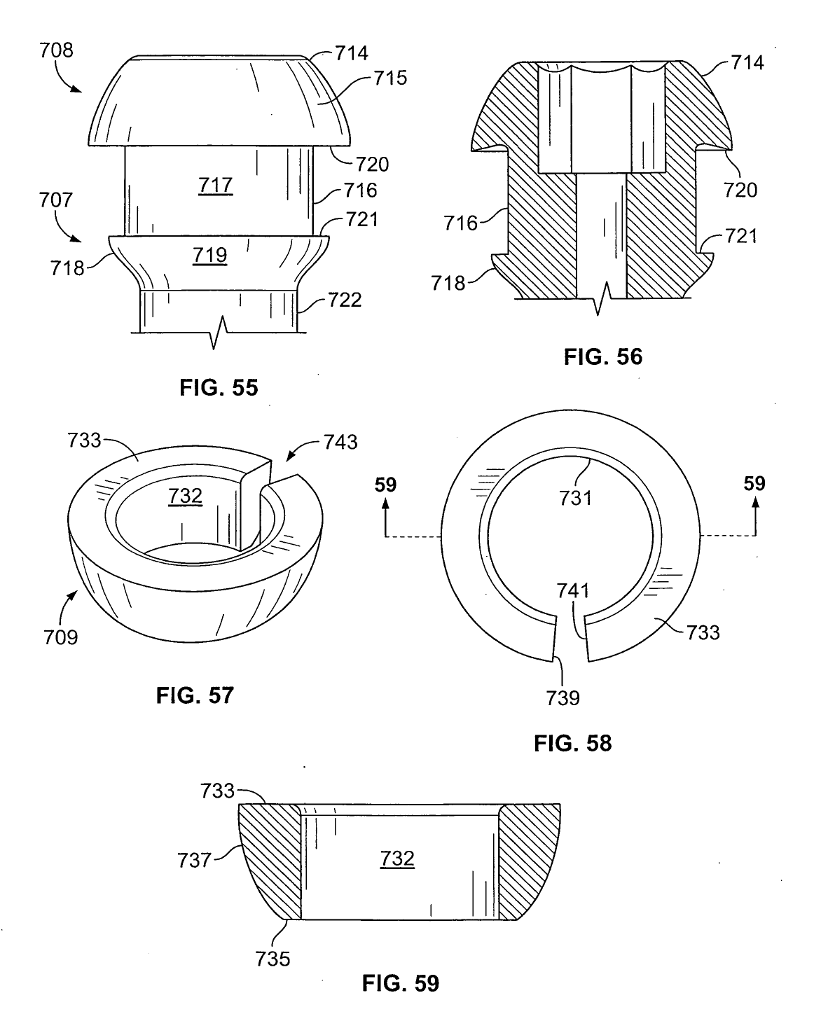

Referring to FIGS. 55-61 there is shown a further alternative embodiment of a shank 707 with a modified capture structure 708 and a modified retainer or retainer ring 709 adapted for securement in a receiver 710, similar in construction to receiver 610 and having a receiver cavity 711 and a spherical seating surface 712. The capture structure 708 includes an upper partially spherical portion 714 with a spherical surface 715, an intermediate, cylindrical body portion 716 with a cylindrical outer surface 717, and a lower, partially spherical portion 718 with a spherical surface 719. The upper portion 714 may be described as having a frusto-hemispherical shape. The diameter of the upper partially spherical portion 714 at its lower edge or widest point is wider than the diameter of the lower cylindrical body portion 716 forming an overhanging, peripheral upper abutment surface 720 that extends transverse to a longitudinal axis of the shank 707 and is preferably planar. The diameter of the lower spherical portion 718 is also wider than the diameter of the cylindrical body portion 716 forming an radially outward projecting lower abutment surface 721 that extends transverse to the longitudinal axis of the shank 707 and is preferably planar.

The capture structure 708 is formed on the shank 707 above a neck 722 which extends above a threaded body 723. The neck 722 is generally the same diameter as or slightly smaller in diameter than the threaded body 723. The lower spherical portion 718 of the capture structure 708 projects radially outward from the neck 722 at its upper end.

Retainer 709 generally comprise a split ring with a central bore 731, defined by an inner cylindrical wall 732, upper surface 733, lower surface 735, partially spherical outer surface 737 and inwardly facing end surfaces 739 and 741 defining a gap therebetween in the retainer ring 710. The retainer 709 may be described as frusto-hemispherical in shape. In the embodiment shown, the upper and lower surfaces 733 and 735 are shown as planar. It is foreseen that the upper surface 735 may include a peripheral, upstanding rim or a downwardly and outwardly curved or sloping chamfer.

The radius of curvature of the spherical outer surface 737 of the retainer 709 and of the spherical outer surface 719 of the lower spherical portion 718 of capture structure 709 match or closely approximate the radius of curvature of the spherical seating surface 712 in receiver cavity 711. In the embodiment shown, the radius of curvature of the spherical surface 715 of the upper spherical portion 714 of capture structure 708 is smaller than the radius of curvature of the spherical outer surface 719 of the lower spherical portion 718.

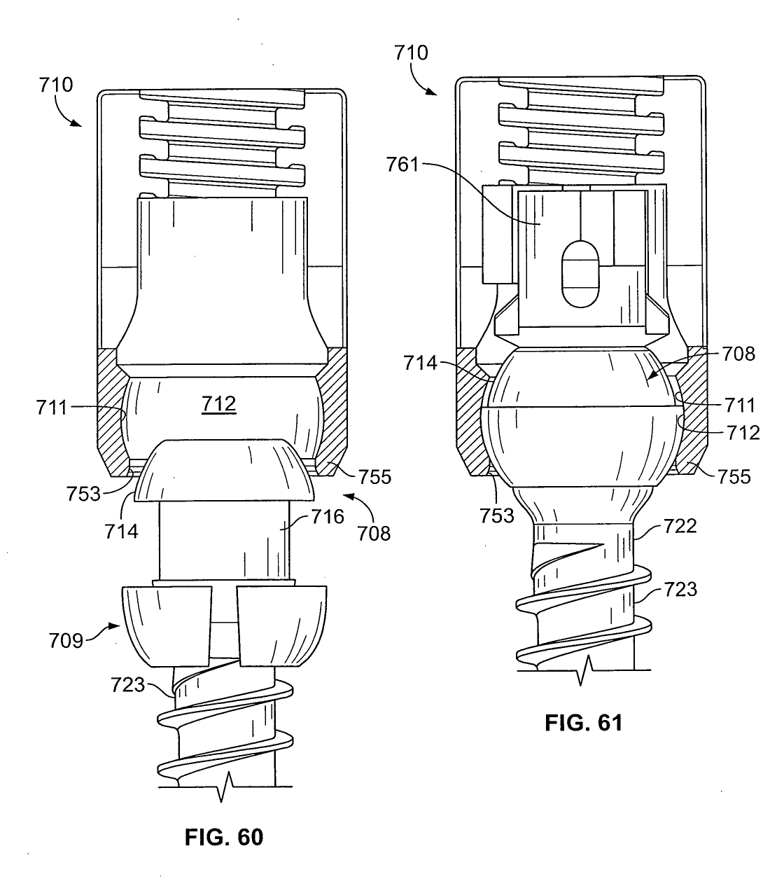

The capture structure 708 on shank 707 and the retainer 709 may be uploaded into the receiver cavity 711 of receiver 710 in a manner similar to that described for the previous embodiments. Referring to FIG. 60, retainer 709 is first secured around neck 722 of shank 707 and compressed until the outer diameter of retainer 709 is smaller than an inner diameter of a bore 753 extending through a neck 755 of receiver 710 in communication with receiver cavity 711. The receiver 710 is then advanced over the capture structure 708 and neck 722 of the shank 707 until the capture structure 708 and the retainer 709 are within the receiver cavity 711. The retainer 709 is allowed to expand to its normal, uncompressed state within the cavity 711.

The shank 707 is then drawn downward, compressing the retainer 709 between the spherical seating surface 712 within the receiver 710 and the spherical outer surface 719 of the lower spherical portion 718 of the capture structure 708. As the shank 707 is drawn further downward relative to the receiver 710, the split retainer 709 expands around the lower spherical portion 718 until the lower abutment surface 721 of capture structure 708 extends just past the lower surface 735 of the retainer 709. The retainer 709 then springs back to an un-expanded state surrounding the intermediate cylindrical body 716 of the capture structure 708. In this configuration, the retainer lower surface 735 extends in closely spaced relation or abuts the lower abutment surface 721 of the capture structure 708 and the retainer upper surface 733 extends in closely spaced relation or abuts the overhanging abutment surface 720 of the capture structure 708, preventing further longitudinal sliding of the retainer 709 relative to the capture structure 708 and the shank 707.

Having the same radius of curvature, the spherical surface 719 of the lower portion 718 of capture structure 708 and the spherical outer surface 737 of the retainer 709 both are supported by and slide relative to the spherical seating surface 712 to permit selected orientation of the shank 707 relative to the receiver 710 prior to securing or fixing the relative position of the receiver 710 to the shank 707. With reference to FIG. 61, a compression insert 761, rod 763 and closure top 765 are loaded into the receiver 710 in a manner the same or substantially similar as previously described herein with respect to the insert 414, rod 421 and closure top 418 of the assembly 401. Tightening of the closure top 765 tp compress the rod 763 against the compression insert 761 and the insert 761 against the upper portion 714 of the capture structure 708 fixes the position of the receiver 710 relative to the shank 707.

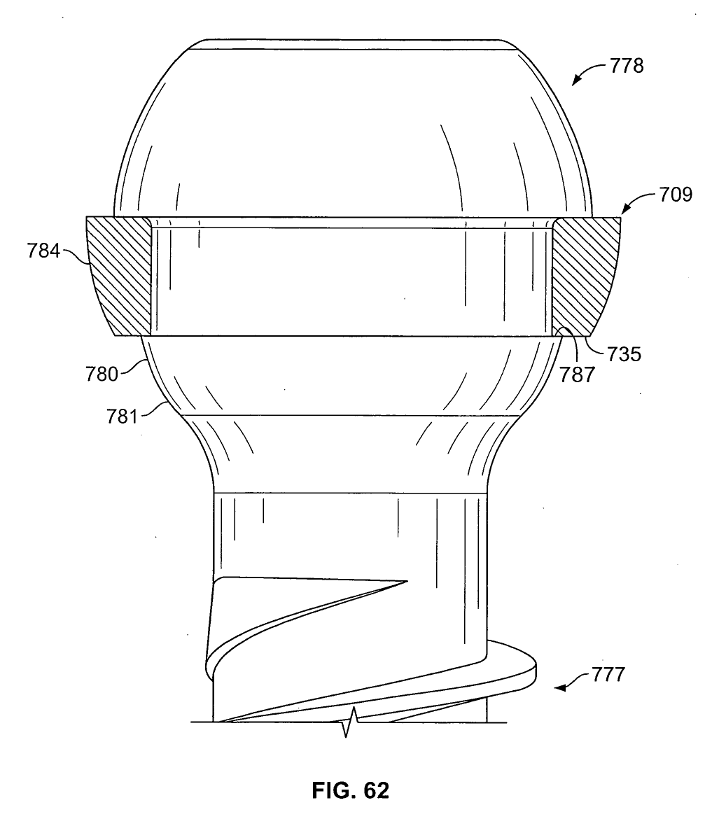

Referring to FIG. 62, there is shown a modified version of a shank 777 and attached capture structure 778 with a retainer ring 709, shown in cross-section, secured around the capture structure 778. Capture structure 778 is similar to capture structure 708, except that the radius of curvature of a spherical surface 780 of a lower spherical portion 781 is smaller than the radius of curvature of an outer spherical surface 784 of retainer 709. The radius of curvature of the spherical surface 780 of lower spherical portion 781 is then also smaller than the radius of curvature of the spherical seating surface 712 of receiver 710 such that the lower spherical portion 781 of capture structure 708 does not abut or seat on the spherical seating surface 712. Because the diameter of the lower spherical portion 781 is reduced, the extent that the split retainer 709 must expand to slide over the lower spherical portion 781 is reduced. Although the diameter of the lower spherical portion 781 is reduced, the lower spherical portion 781 still extends past the intermediate cylindrical body portion 716 enough to form a lower abutment surface 787 that is sufficiently wide to engage the lower surface 735 of the retainer 709 and prevent the retainer 709 from sliding back down the shank 777.

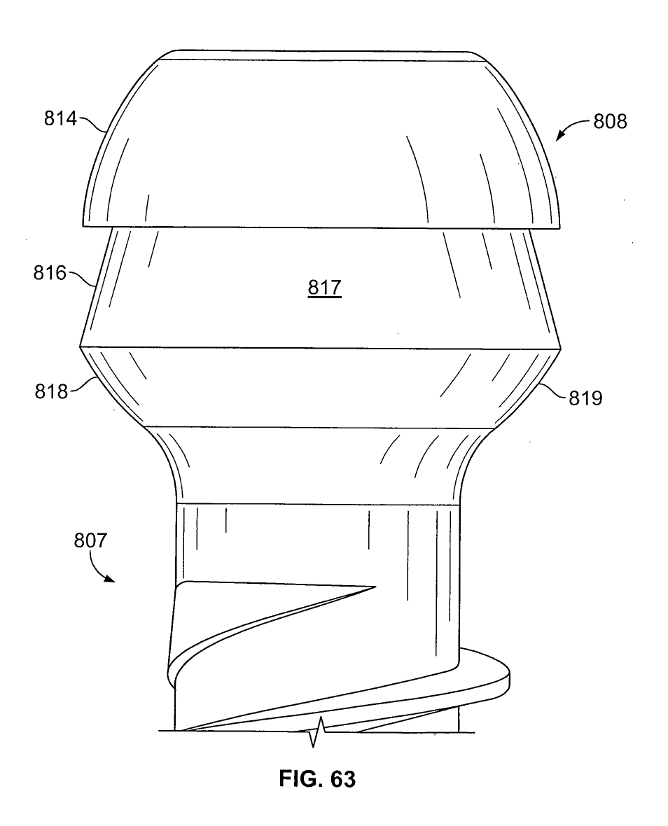

FIGS. 63-65 disclose a modified version of the shank and retainer as shown in FIGS. 55-61 comprising shank 807 with capture structure 808 and modified retainer 809. The capture structure 808 includes upper and lower spherical portions 814 and 818 which are similar in configuration to upper and lower spherical portions 714 and 718 of capture structure 708. However, an intermediate portion 816 of capture structure 808 is conical or frusto-conical instead of cylindrical as with the intermediate portion 716 of capture structure 708. An outer surface 817 of the frusto-conical intermediate portion 816 slopes inward from the lower spherical portion 818 to the upper spherical portion 814. A lower edge of the outer surface 817 of intermediate portion 816 extends flush with an upper edge of an outer surface 819 of the lower spherical portion 818 of capture structure 808. However, it is foreseen that the lower spherical portion 818 may be wider than the intermediate frusto-conical portion 816 at their interface to form an upwardly facing abutment surface.

The split retainer 809 is similar in construction as retainer 709 except that an inner surface 832 thereof slopes inwardly from a lower surface 835 and comes to a point or edge with the outer spherical surface 837 thereof. The radius of curvature of the outer spherical surface 837 of retainer 809 and of the outer surface 819 of the lower spherical portion 818 of the capture structure 808 match the radius of curvature of the spherical seating surface 712 in an associated receiver 710.

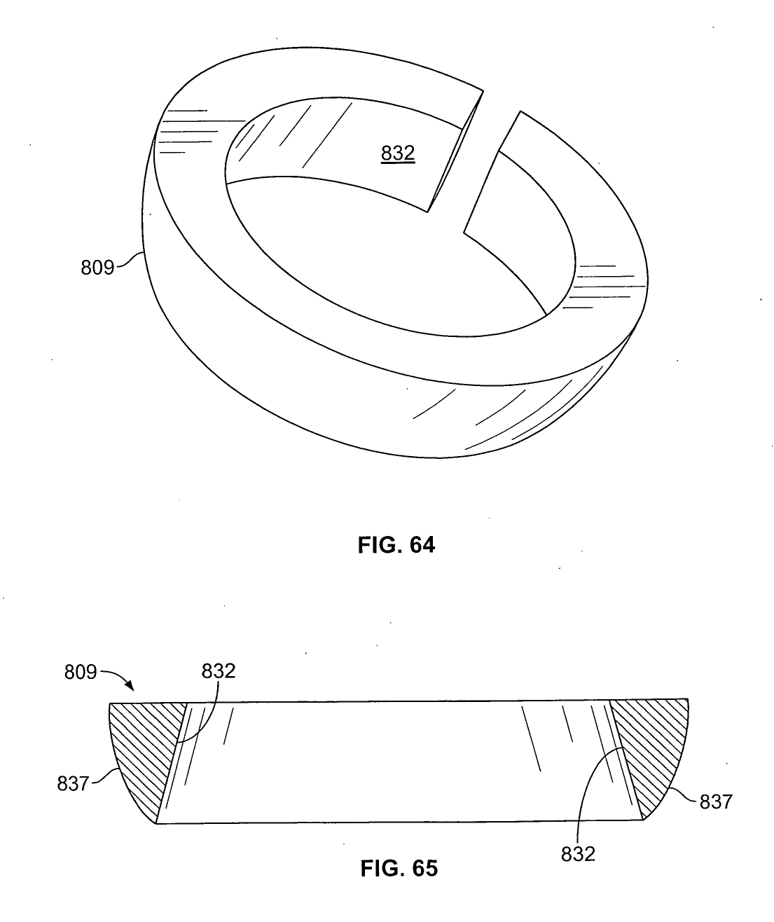

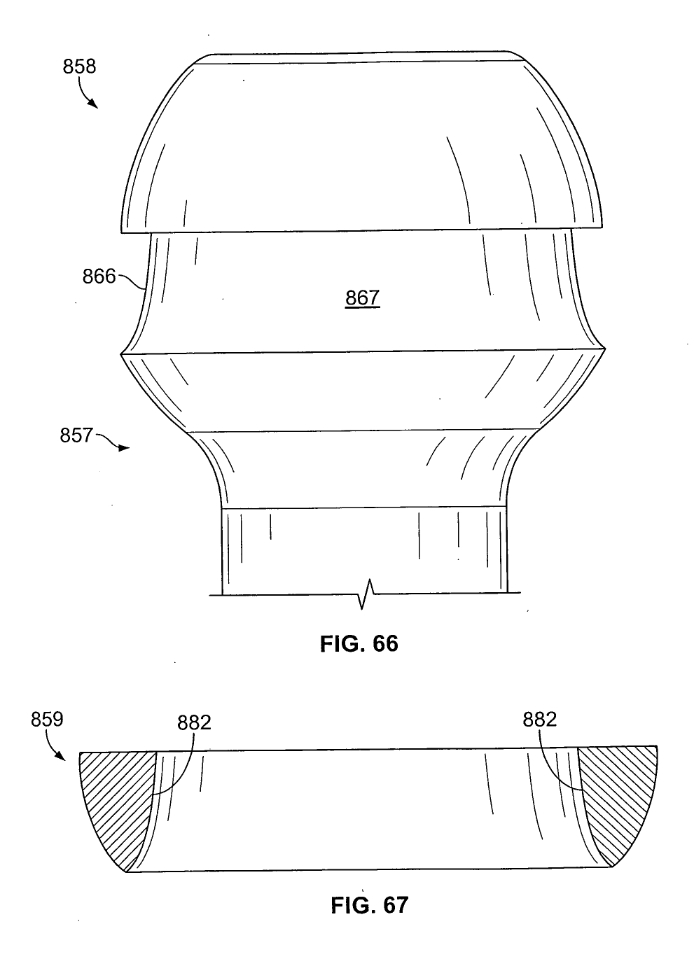

FIGS. 66 and 67 disclose a modified version of the shank and retainer as shown in FIGS. 63 and 64 comprising shank 857 with capture structure 858 and modified retainer 859. An outer surface 867 of an intermediate portion 866 of the capture structure 858 is curvate and an inner surface 882 of retainer 859 has a mating curvate geometry.

It is to be understood that while certain forms of the present invention have been illustrated and described herein, it is not to be limited to the specific forms or arrangement of parts described and shown.

What is claimed and desired to be secured by Letters Patent is as follows:

1. A polyaxial bone anchor comprising:
a) a shank having a body for fixation to a bone and an upper portion, the body and upper portion being generally aligned along an axis of rotation thereof, the upper portion having an upper surface, a first section and a second section, the first section having a first width measured perpendicular to the axis, the second section having a second width measured perpendicular to the axis, the second width being different than the first width, the second section being frusto-conical and being disposed between the body and the first section;
b) a receiver having a top portion and a base, the receiver top portion defining an open channel, the base having a seating surface partially defining a cavity, the channel communicating with the cavity, the cavity communicating with an exterior of the base through an opening sized and shaped to receive the shank upper portion therethrough; and
c) a retainer having a through slit and an internal surface sized and shaped to frictionally engage and mate with the shank second section, the shank upper portion and the retainer being in non-swivelable fixed axial relation to one another, both the upper portion and the retainer being in swivelable relation within the receiver, providing selective angular positioning of the shank with respect to the receiver, the retainer being in slidable engagement with the receiver seating surface.

2. The bone anchor of claim 1 further comprising an external drive feature extending above the shank upper surface and being integral therewith.

3. The bone anchor of claim 1 further comprising an annular rib projecting radially outwardly from at least the shank second section and into a mating annular groove of the retainer.

4. The bone anchor of claim 1 further comprising an overhanging portion extending from the shank first section downwardly toward the second section and into a grooved surface of the retainer.

5. The bone anchor of claim 1 wherein the shank first section has a lower annular ledge in frictional engagement with an annular surface of the retainer.

6. The bone anchor of claim 1 wherein the shank second section widens in a direction toward the shank upper surface.

7. The bone anchor of claim 1 wherein the shank second section widens in a direction toward the shank body.

8. The bone anchor of claim 1 further comprising a compression insert disposed in the receiver, the insert having a mating surface exclusively frictionally engageable with the upper surface of the shank upper portion.

9. The bone anchor of claim 8 wherein the compression insert mating surface is concave and the shank upper surface is convex.

10. The bone anchor of claim 8 wherein the shank upper portion has a tool engagement formation formed in the upper surface adapted for non-slip engagement by a tool for driving the bone screw shank body into bone.

11. The bone anchor of claim 1 wherein the receiver seating surface is at least partially spherical and the retainer has an outer surface that is at least partially spherical.

12. The bone anchor of claim 1 wherein the retainer is sized and shaped to be bottom-loadable into the receiver.

13. The bone anchor of claim 1 wherein the retainer comprises first and second spaced ends, the retainer being compressible and expandable with the first and second ends being movable toward and away from one another.

14. A polyaxial bone screw assembly comprising:
(a) a shank having a body for fixation to a bone and an upper portion, the upper portion having a top surface and an upper side surface, the upper surface having a first portion and an adjacent second portion, the first portion being frusto-conical in shape; the shank being elongate and having a central axis;
(b) a receiver having a top portion and a base, the receiver top portion defining an open channel, the base having a seating surface partially defining a cavity, the channel communicating with the cavity, the cavity communicating with an exterior of the base through an opening sized and shaped to receive the shank upper portion therethrough; and
(c) a retainer having a top surface substantially spaced from the shank top surface of the shank upper portion, the retainer defining a through slit and having an internal surface with a first inner surface portion sized and shaped to substantially mate with an frictionally engage the shank at the first surface portion and a second surface sized and shaped to substantially frictionally engage the second shank surface portion, the shank upper portion and the retainer being in fixed axial relation to one another, both the upper portion and the retainer being in swivelable relation within the receiver, providing selective angular positioning of the shank with respect to the receiver, the retainer having an outer surface in slidable engagement with the receiver seating surface.

15. The assembly of claim 14 wherein the shank second portion is curvate.

16. The assembly of claim 14 wherein the shank second portion is an overhang disposed at an acute angle with respect to the first portion.

17. The assembly of claim 14 wherein the shank second portion is a planar annular surface disposed perpendicular to the first portion.

18. A polyaxial bone screw assembly comprising:
(a) a shank having a body for fixation to a bone and an upper portion, the upper portion having a top surface and an upper surface, the upper surface having a first portion and an adjacent second portion, the first portion being frusto-conical in shape;
(b) a receiver having a top portion and a base, the receiver top portion defining an open channel, the base having a seating surface partially defining a cavity, the channel communicating with the cavity, the cavity communicating with an exterior of the base through an opening sized and shaped to receive the shank upper portion therethrough;
(c) a compression insert disposed in the receiver, the insert having a mating surface exclusively frictionally engageable with the upper surface of the shank upper portion; and
(d) a retainer having a top surface substantially spaced from the shank top surface of the shank upper portion, the retainer defining a through slit and having a first inner surface portion sized and shaped to substantially frictionally engage the shank at the shank first surface portion and a retainer second surface sized and shaped to substantially frictionally engage the second shank surface portion, the shank upper portion and the retainer being in fixed axial relation to one another, both the upper portion and the retainer being in swivelable relation within the receiver, providing selective angular positioning of the shank with respect to the receiver, the retainer having an outer surface in slidable engagement with the receiver seating surface.

19. The assembly of claim 18 wherein the retainer second surface partially defines a groove.

20. The assembly claim 18 wherein the retainer second surface is planar and annular.

21. The assembly of claim 18 wherein the retainer second surface is radiused.

22. The assembly of claim 18 wherein the retainer second surface partially defines a v-shaped groove in the top surface of the retainer.

23. The assembly of claim 18 wherein the retainer second surface is the retainer top surface.

24. A polyaxial bone screw assembly comprising:
(a) a shank having a body for fixation to a bone and an upper portion, the upper portion having a top surface and an outer frusto-conical mating surface;
(b) a receiver having a top portion and a base, the receiver top portion defining an open channel, the base having a seating surface partially defining a cavity, the channel communicating with the cavity, the cavity communicating with an exterior of the base through an opening sized and shaped to receive the shank upper portion therethrough;
(c) a compression insert disposed in the receiver, the insert having a mating surface exclusively frictionally engageable with the top surface of the shank upper portion; and
(d) a retainer having a top surface substantially spaced from the shank top surface of the shank upper portion, the retainer defining a through slit and having an inner surface sized and shaped to substantially frictionally engage the shank at the shank frusto-conical outer mating surface, the shank upper portion and the retainer being in fixed axial relation to one another, both the upper portion and the retainer being in swivelable relation within the receiver, providing selective angular positioning of the shank with respect to the receiver, the retainer having an outer surface in slidable engagement with the receiver seating surface.
